(19) United States
(12) Patent Application Publication
(10) Pub. No.: US 2017/0101470 A1
(43) Pub. Date: Apr. 13, 2017

(54) Use of Anti-MCAM Antibodies for Treatment or Prophylaxis of Giant Cell Arteritis, Polymyalgia Rheumatica or Takayasus Arteritis

(71) Applicant: PROTHENA BIOSCIENCES LIMITED, Dublin (IE)

(72) Inventors: Yue Liu, Foster City, CA (US); Patrick Garidel, Ingelheim am Rhein (DE); Andreas Langer, Ingelheim am Rhein (DE); Kenneth Flanagan, Alameda, CA (US)

(21) Appl. No.: 15/268,178

(22) Filed: Sep. 16, 2016

Related U.S. Application Data
(60) Provisional application No. 62/219,599, filed on Sep. 16, 2015.

(51) Int. Cl. C07K 16/28; C07K 16/30; A61K 47/26; A61K 39/395; A61K 47/22

(52) U.S. Cl. CPC C07K 16/2803; A61K 39/39558; A61K 47/22; A61K 47/26; C07K 16/3092; C07K 2317/56; C07K 2317/52; C07K 2317/24; C07K 2317/34; C07K 2317/565; C07K 2317/92

(57) ABSTRACT

The invention provides anti-MCAM antibodies that inhibit the ability of human MCAM to bind a laminin alpha-4 chain and pharmaceutical compositions and pharmaceutical formulations incorporating the same for use in treatment or prophylaxis of giant cell arteritis, polymyalgia rheumatica (PMR) or Takayasu's arteritis, methods of generating such antibodies, and their use in the manufacture of medicaments for treatment of neuroinflammatory disease, autoimmune disease, or cancer.

### CROSS-REFERENCES TO RELATED APPLICATIONS

This application claims the benefit under 35 USC 119(e) of U.S. Provisional Application No. 62/219,599 filed Sep. 16, 2015, the disclosure of which is herein incorporated by reference in its entirety. U.S. Provisional Application No. 61/952,116, filed Mar. 12, 2014, U.S. Provisional Application No. 61/952,833, filed Mar. 13, 2014, U.S. Provisional Application No. 62/023,724, filed Jul. 11, 2014, and U.S. Provisional Application No. 62/068,419, filed Oct. 24, 2014 and U.S. Ser. No. 14/656,596 filed Mar. 12, 2015 discloses related subject matter and is each incorporated in its entirety herein for all purposes.

### REFERENCE TO A SEQUENCE LISTING, A TABLE, OR A COMPUTER PROGRAM LISTING

The Sequence Listing written in file 480267_SEQLST.txt, created on Sep. 15, 2016, for “USE OF ANTI-MCAM ANTIBODIES FOR TREATMENT OR PROPHYLAXIS OF GIANT CELL ARTERITIS, POLYMYALGIA RHEUMATICA OR TAKAYASU'S ARTERITIS” is 158 kilobytes. The information contained in this file is hereby incorporated by reference.

### BACKGROUND

A subset of CD4+ T cells, termed TH17 cells (T helper 17 cells), has been implicated in the pathogenesis of a number of autoimmune diseases, particularly those neuroinflammatory conditions involving CNS infiltration of T cells, such as multiple sclerosis and the animal model, experimental autoimmune encephalomyelitis (EAE). TH17 cells have been reported to secrete a number of select cytokines including IL-17 and IL-22. TH17 cells have been reported to undergo specific recruitment and infiltration of tissue. MCAM has been reported to be expressed on TH17 cells and to bind laminin alpha-4 as a ligand.

### SUMMARY OF THE CLAIMED INVENTION

The invention provides antibodies, compositions, formulations and the like described below and throughout the specification for use in treatment or prophylaxis of giant cell arteritis, polymyalgia rheumatica (PMR) or Takayasu's arteritis. Corresponding claims in method or Swiss style are also provided.

Antibodies, and formulations for such uses and methods are described as follows. Some antibodies comprise a mature heavy chain variable region comprising the three Kabat CDRs of SEQ ID NO:161 except that position 32 (Kabat numbering) can be N, S, or Q, and position 33 (Kabat numbering) can be G or A and wherein position 1 (Kabat numbering) is occupied by E, and a mature light chain variable region comprising the three Kabat CDRs of SEQ ID NO:123. In some antibodies, the mature heavy chain variable region is at least 90% identical to SEQ ID NO:161, and the mature light chain variable region is at least 90% identical to SEQ ID NO:123. Some such antibodies are humanized antibodies. In some of such antibodies, the mature heavy chain variable region is at least 95%, 96%, 97%, 98% or 99% identical to SEQ ID NO:161 and the mature light chain variable region is at least 98% or 99% identical to SEQ ID NO:123. In some of such antibodies, the mature heavy chain variable region is at least 95%, 96%, 97%, 98% or 99% identical to SEQ ID NO:161 and the mature light chain variable region is at least 95%, 96%, 97%, 98% or 99% identical to SEQ ID NO:123. In some such antibodies, the mature heavy chain variable region has the amino acid sequence of SEQ ID NO:157, SEQ ID NO:158, SEQ ID NO:159, SEQ ID NO:160, or SEQ ID NO:161, and wherein the mature light chain variable region is at least 95% identical to SEQ ID NO:123. In some such antibodies, the mature heavy chain variable region is at least 95% identical to SEQ ID NO:161 and the mature light chain variable region has the amino acid sequence of SEQ ID NO:121, SEQ ID NO:122, or SEQ ID NO:123. In some such antibodies, the mature heavy chain variable region has the amino acid sequence of SEQ ID NO:157, SEQ ID NO:158, SEQ ID NO:159, SEQ ID NO:160, or SEQ ID NO:161, and the mature light chain variable region has the amino acid sequence of SEQ ID NO:121, SEQ ID NO:122, or SEQ ID NO:123. In some such antibodies, the mature heavy chain variable region has the amino acid sequence of SEQ ID NO:161, and the mature light chain variable region has the amino acid sequence of SEQ ID NO:123. In some such antibodies, the heavy chain constant region has the amino acid sequence of SEQ ID NO:171 or 172 and/or the light chain constant region has the amino acid sequence of SEQ ID NO:168.

Some anti-MCAM antibodies bind to human MCAM (SEQ ID NO:11) at an epitope including amino acid residue 141. In some antibodies, the epitope comprises amino acid residue 145. In some antibodies, the epitope comprises at least five contiguous amino acids residues of human MCAM including amino acid residue 141. In some such antibodies, the antibody is not an antibody selected from the group consisting of:

(a) clone 15 having a mature heavy chain variable region corresponding to SEQ ID NO:18 and a mature light chain variable region corresponding to SEQ ID NO:13;

(b) clone 17 having a mature heavy chain variable region corresponding to SEQ ID NO:7 and a mature light chain variable region corresponding to SEQ ID NO:2;

(c) 1174.1.3 having a mature heavy chain variable region corresponding to SEQ ID NO:35 and a mature light chain variable region corresponding to SEQ ID NO:30;

(d) 1414.1.2 having a mature heavy chain variable region corresponding to SEQ ID NO:45 and a mature light chain variable region corresponding to SEQ ID NO:40;

(e) 1415.1.1 having a mature heavy chain variable region corresponding to SEQ ID NO:55 and a mature light chain variable region corresponding to SEQ ID NO:50;

(f) 1749.1.3 having a mature heavy chain variable region corresponding to SEQ ID NO:65 and a mature light chain variable region corresponding to SEQ ID NO:60;

(g) 2120.4.19 having a mature heavy chain variable region corresponding to SEQ ID NO:77 and a mature light chain variable region corresponding to SEQ ID NO:70;

(h) 2107.4.10 having a mature heavy chain variable region corresponding to SEQ ID NO:89 and a mature light chain variable region corresponding to SEQ ID NO:84: and

(i) an antibody comprising CDRs substantially from the monoclonal antibodies 1174.1.3, 1414.1.2, 1415.1.1, 1749.1.3, 2120.4.19, and 2107.4.10. In some such antibodies, the antibody is monoclonal. In some such antibodies, the antibody is chimeric, humanized, veneered, or human.

In some such antibodies, the antibody is not an antibody selected from the group consisting of:

(a) clone 15 having a mature heavy chain variable region corresponding to SEQ ID NO:18 and a mature light chain variable region corresponding to SEQ ID NO:13;

(b) clone 17 having a mature heavy chain variable region corresponding to SEQ ID NO:7 and a mature light chain variable region corresponding to SEQ ID NO:2;

(c) 1174.1.3 having a mature heavy chain variable region corresponding to SEQ ID NO:35 and a mature light chain variable region corresponding to SEQ ID NO:30;

(d) 1414.1.2 having a mature heavy chain variable region corresponding to SEQ ID NO:45 and a mature light chain variable region corresponding to SEQ ID NO:40;

(e) 1415.1.1 having a mature heavy chain variable region corresponding to SEQ ID NO:55 and a mature light chain variable region corresponding to SEQ ID NO:50;

(f) 1749.1.3 having a mature heavy chain variable region corresponding to SEQ ID NO:65 and a mature light chain variable region corresponding to SEQ ID NO:60;

(g) 2120.4.19 having a mature heavy chain variable region corresponding to SEQ ID NO:77 and a mature light chain variable region corresponding to SEQ ID NO:70, 71, or 72;

(h) 2107.4.10 having a mature heavy chain variable region corresponding to SEQ ID NO:89 and a mature light chain variable region corresponding to SEQ ID NO:82 or 84: and

(i) an antibody comprising CDRs substantially from the monoclonal antibodies 1174.1.3, 1414.1.2, 1415.1.1, 1749.1.3, 2120.4.19, and 2107.4.10. In some such antibodies, the antibody is monoclonal. In some such antibodies, the antibody is chimeric, humanized, veneered, or human.

Also provided is a pharmaceutical composition comprising any of the above-mentioned antibodies.

Some pharmaceutical formulations comprising (a) any antibody described herein present at a concentration within the range from about 1 mg/mL to about 100 mg/mL; (b) a buffer, such as histidine buffer, present at a concentration within the range from about 10 mM to about 30 mM; (c) a sugar and/or polyol, such as sucrose or trehalose, present at a concentration within the range from about 200 mM to about 260 mM; and (d) a surfactant, such as polysorbate 20, present at a concentration within the range from about 0.005% to about 0.05% by weight; wherein the formulation is characterized by a pH within the range from about 5.5 to about 7.

An exemplary pharmaceutical formulation comprises (a) any antibody described herein, wherein the antibody is present at a concentration of about 40 mg/mL; (b) histidine buffer present at a concentration of about 20 mM; (c) sucrose present at a concentration of about 220 mM; (d) polysorbate 20 present at a concentration of about 0.02%; and (e) a pH of about 6.0.

Another exemplary pharmaceutical formulation comprises (a) any antibody described herein, wherein the antibody is present at a concentration of about 40 mg/mL; (b) histidine buffer present at a concentration of about 20 mM; (c) trehalose present at a concentration of about 220 mM; (d) polysorbate 20 present at a concentration of about 0.02%; and (e) a pH of about 6.5.

Some formulations further comprise a bulking agent, are sterile, and/or are stable on freezing and thawing. In some formulations, at least 65% of antibody appears as a single peak on hydrophobic interaction chromatography after storage for at least 30 days at 38-42° C. and/or after storage for at least 3 months at 38-42° C. In some formulations, no more than 5% aggregated protein by weight on high performance size exclusion chromatography after storage for at least 30 days at 38-42° C. and/or after storage for at least 3 months at 38-42° C.

The antibody formulations can be in the form of a lyophilized formulation. For example, a representative lyophilized formulation can comprise: (a) any antibody described herein; (b) histidine buffer; (c) sucrose or trehalose; and (d) polysorbate 20. Lyophilized formulations can comprise about 10 mg to about 40 mg of the antibody and polysorbate 20 at a concentration within the range from about 0.005% to about 0.05% by weight. Following reconstitution, the lyophilized formulations can comprise about 10 mM to about 30 mM histidine buffer and about 200 mM to about 260 mM of sucrose or trehalose. Following reconstitution, the lyophilized formulations yield an aqueous solution having a pH of between about 6 to about 7, such as pH 6.0 or 6.5.

Following reconstitution, an exemplary lyophilized formulation can comprise: (a) any antibody described herein, which is present at a concentration of about 40 mg/mL; (b) histidine buffer present at a concentration of about 20 mM; (c) sucrose present at a concentration of about 220 mM; (d) polysorbate 20 present at a concentration of about 0.2 g/L; and (e) a pH of about 6.0. Such a lyophilized formulation can comprise about 200 mg of the antibody, about 15.5 mg of histidine, about 376 mg sucrose, and about 1 mg polysorbate 20.

Following reconstitution, another exemplary lyophilized formulation can comprise: (a) any antibody described herein, which is present at a concentration of about 40 mg/mL; (b) histidine buffer present at a concentration of about 20 mM; (c) trehalose dihydrate present at a concentration of about 220 mM; (d) polysorbate 20 present at a concentration of about 0.2 g/L; and (e) a pH of about 6.5. Such a lyophilized formulation can comprise about 200 mg of the antibody, about 15.5 mg of histidine, about 416 mg trehalose dihydrate, and about 1 mg polysorbate 20.

The invention further provides an isolated peptide comprising an epitope for binding an anti-MCAM monoclonal antibody, wherein the peptide comprises 5-50 contiguous amino acid residues of human MCAM (SEQ ID NO:11) including amino acid residue 141. In some of these peptides, the peptide is linked to a carrier polypeptide. In some of these peptides, the peptide is combined with an adjuvant for use in treatment or prophylaxis of giant cell arteritis, polymyalgia rheumatica (PMR) or Takayasu's arteritis.

The invention further provides ahumanized 2107 antibody for use in treatment or prophylaxis of giant cell arteritis, polymyalgia rheumatica (PMR) or Takayasu's arteritis. Optionally, the antibody comprises a mature heavy chain variable region of SEQ ID NO:178 or 179. Optionally, the antibody further comprises a mature light chain variable region of SEQ ID NO:98.

### BRIEF DESCRIPTION OF THE DRAWINGS

FIG. 1 depicts the identification of critical clones. The mean 2120.4.19 binding value plotted as a function of its mean surface expression value (gray diamonds). Thresholds of <30% monoclonal antibody reactivity and >50% mouse sera binding were applied to identify clones (black diamonds) that were negative for antibody binding but positive for surface expression

FIGS. 2A-C. FIG. 2A a homology model of human MCAM, represented by a ribbon diagram. FIG. 2B depicts a partial alignment of human BCAM, human MCAM, and mouse MCAM sequences indicating residues of interest at position 141 (1141) and position 145 (P145) of human MCAM. FIG. 2C depicts a ribbon diagram depicting the location and exposure of the 1141 and P145 residues of human MCAM.

FIGS. 3A & B. FIG. 3A shows the alignment of sequences of the variable heavy chains for the following: rat 2120.4.19 anti-MCAM antibody (2120.4.19.6_VH_topo_pro; SEQ ID NO:114); 2120 VH1 humanized anti-MCAM antibody (h2120VH1; SEQ ID NO:115); 2120 VH2 humanized anti-MCAM antibody (h2120VH2; SEQ ID NO:116); 2120 VH3 humanized anti-MCAM antibody (h2120VH3; SEQ ID NO:117); 2120 VH4 humanized anti-MCAM antibody (h2120VH4; SEQ ID NO:118); 2120 VH5 humanized anti-MCAM antibody (h2120VH5; SEQ ID NO:119); and heavy chain human variable AF062133 IGHV2-26*01 sequence used as the framework donor (AF062133_VH; SEQ ID NO:108). Kabat numbering is used and hypervariable regions (HVRs) grafted from the rat 2120.4.19.6 antibody to the variable heavy chain variable AF062133 IGHV2-26*01 framework are boxed. The S30T, I37V, L48I and K71R mutations combined with (i) mutations of the boxed N/D residues in CDR-H1, e.g., N32S (VH3); N32Q (VH4); or G33A (VH5)), provides an N deamidation mutant. The bolded amino acid residues in the humanized antibody sequences differ from the corresponding residues in the rat antibody sequence. The position of canonical and interface amino acid residues that may affect CDR contact or CDR structure are indicated by an asterisk. Residues where mutations were focused due to the presence of N-deamination sites or N-glycosylation sites are shown in the bracketed box.

FIG. 3B shows the alignment of sequences of the variable light chains for the following: rat 2120.4.19.6 anti-MCAM antibody (2120.4.19.6_VL_topo_pro; SEQ ID NO:120); 2120 VL1 humanized anti-MCAM antibody (h2120VL1 SEQ ID NO:121); 2120 VL2 humanized anti-MCAM antibody (h2120VL2 SEQ ID NO:122); 2120 VL3 humanized anti-MCAM antibody (h2120VL3 SEQ ID NO:123); and light chain human variable X84343 IGKV2-26*01 sequence used as the framework donor (X84343_VL SEQ ID NO:124). Kabat numbering is used and hypervariable regions (HVRs) grafted from the rat 2120.4.19.6 antibody to the variable light chain variable X84343 IGKV2-26*01 framework are boxed. The bolded amino acid residues in the humanized antibody sequences differ from the corresponding residues in the rat antibody sequence. The position of canonical and interface amino acid residues that may affect CDR contact or CDR structure are indicated by an asterisk.

FIG. 4A shows the alignment of sequences of the mature heavy chain variable regions for the following: rat 2120.4.19 anti-MCAM antibody (2120.4.19.6_VH_topo_pro; SEQ ID NO:114); 2120 VH1.Q1E humanized anti-MCAM antibody (h2120VH1.Q1E; SEQ ID NO:157); 2120 VH2.Q1E humanized anti-MCAM antibody (h2120VH2.Q1E; SEQ ID NO:158); 2120 VH3.Q1E humanized anti-MCAM antibody (h2120VH3.Q1E; SEQ ID NO:159); 2120 VH4.Q1E humanized anti-MCAM antibody (h2120VH4.Q1E; SEQ ID NO:160); 2120 VH5.Q1E humanized anti-MCAM antibody (h2120VH5.Q1E; SEQ ID NO:161); and heavy chain human variable AF062133 IGHV2-26*01 sequence used as the framework donor (AF062133_VH; SEQ ID NO:108). Kabat numbering is used and hypervariable regions (HVRs) grafted from the rat 2120.4.19.6 antibody to the variable heavy chain variable AF062133 IGHV2-26*01 framework are boxed. The position Q1E substitution is outlined by a box.

FIG. 4B shows the alignment of sequences of the variable light chains for the following: rat 2120.4.19.6 anti-MCAM antibody (2120.4.19.6_VL_topo_pro; SEQ ID NO:120); 2120 VL1 humanized anti-MCAM antibody (h2120VL1; SEQ ID NO:121); 2120 VL2 humanized anti-MCAM antibody (h2120VL2; SEQ ID NO:122); 2120 VL3 humanized anti-MCAM antibody (h2120VL3; SEQ ID NO:123); and light chain human variable X84343 IGKV2-26*01 sequence used as the framework donor (X84343_VL SEQ ID NO:124). Kabat numbering is used and hypervariable regions (HVRs) grafted from the rat 2120.4.19.6 antibody to the variable light chain variable X84343 IGKV2-26*01 framework are boxed.

### BRIEF DESCRIPTION OF THE SEQUENCES

SEQ ID NO:1 is the nucleic acid sequence encoding the mature light chain variable region of antibody clone 17.

SEQ ID NO:2 is the amino acid sequence of the mature light chain variable region of antibody clone 17.

SEQ ID NO:3 is the amino acid sequence of CDRL1 of the antibody clone 17.

SEQ ID NO:4 is the amino acid sequence of CDRL2 of the antibody clone 17.

SEQ ID NO:5 is the amino acid sequence of CDRL3 of the antibody clone 17.

SEQ ID NO:6 is the nucleic acid sequence encoding the mature heavy chain variable region of antibody clone 17.

SEQ ID NO:7 is the amino acid sequence of the mature heavy chain variable region of antibody clone 17.

SEQ ID NO:8 is the amino acid sequence of CDRH1 of the antibody clone 17.

SEQ ID NO:9 is the amino acid sequence of CDRH2 of the antibody clone 17.

SEQ ID NO:10 is the amino acid sequence of CDRH3 of the antibody clone 17.

SEQ ID NO:11 is the amino acid sequence of human MCAM Accession No. CAA48332.

SEQ ID NO:12 is the nucleic acid sequence encoding the mature light chain variable region of antibody clone 15.

SEQ ID NO:13 is the amino acid sequence of the mature light chain variable region of antibody clone 15.

SEQ ID NO:14 is the amino acid sequence of CDRL1 of the antibody clone 15.

SEQ ID NO:15 is the amino acid sequence of CDRL2 of the antibody clone 15.

SEQ ID NO:16 is the amino acid sequence of CDRL3 of the antibody clone 15.

SEQ ID NO:17 is the nucleic acid sequence encoding the mature heavy chain variable region of antibody clone 15.

SEQ ID NO:18 is the amino acid sequence of the mature heavy chain variable region of antibody clone 15.

SEQ ID NO:19 is the amino acid sequence of CDRH1 of the antibody clone 15.

SEQ ID NO:20 is the amino acid sequence of CDRH2 of the antibody clone 15.

SEQ ID NO:21 is the amino acid sequence of CDRH3 of the antibody clone 15.

SEQ ID NO:22 is the amino acid sequence of human MCAM domain 1 (residues 19-129).

SEQ ID NO:23 is the amino acid sequence of human MCAM domain 2 (residues 139-242).

SEQ ID NO:24 is the amino acid sequence of human MCAM domain 3 (residues 244-321).

SEQ ID NO:25 is the amino acid sequence of human MCAM domain 4 (residues 355-424).

SEQ ID NO:26 is the amino acid sequence of human MCAM domain 5 (residues 430-510).

SEQ ID NO:27 is the amino acid sequence of an α4-chain isoform of human laminin 411 (Accession No. NP001098676).

SEQ ID NO:28 is the amino acid sequence of an α4-chain isoform of human laminin 411 (Accession No. CAA48332).

SEQ ID NO:29 is the nucleic acid sequence encoding the mature light chain variable region of antibody 1174.1.3.

SEQ ID NO:30 is the amino acid sequence of the mature light chain variable region of antibody 1174.1.3.

SEQ ID NO:31 is the amino acid sequence of CDRL1 of antibody 1174.1.3.

SEQ ID NO:32 is the amino acid sequence of CDRL2 of antibody 1174.1.3.

SEQ ID NO:33 is the amino acid sequence of CDRL3 of antibody 1174.1.3.

SEQ ID NO:34 is the nucleic acid sequence encoding the mature heavy chain variable region of antibody 1174.1.3.

SEQ ID NO:35 is the amino acid sequence of the mature heavy chain variable region of antibody 1174.1.3.

SEQ ID NO:36 is the amino acid sequence of CDRH1 of antibody 1174.1.3.

SEQ ID NO:37 is the amino acid sequence of CDRH2 of antibody 1174.1.3.

SEQ ID NO:38 is the amino acid sequence of CDRH3 of antibody 1174.1.3.

SEQ ID NO:39 is the nucleic acid sequence encoding the mature light chain variable region of antibody 1414.1.2.

SEQ ID NO:40 is the amino acid sequence of the mature light chain variable region of antibody 1414.1.2.

SEQ ID NO:41 is the amino acid sequence of CDRL1 of antibody 1414.1.2.

SEQ ID NO:42 is the amino acid sequence of CDRL2 of antibody 1414.1.2.

SEQ ID NO:43 is the amino acid sequence of CDRL3 of antibody 1414.1.2.

SEQ ID NO:44 is the nucleic acid sequence encoding the mature heavy chain variable region of antibody 1414.1.2.

SEQ ID NO:45 is the amino acid sequence of the mature heavy chain variable region of antibody 1414.1.2.

SEQ ID NO:46 is the amino acid sequence of CDRH1 of antibody 1414.1.2.

SEQ ID NO:47 is the amino acid sequence of CDRH2 of antibody 1414.1.2.

SEQ ID NO:48 is the amino acid sequence of CDRH3 of antibody 1414.1.2.

SEQ ID NO:49 is the nucleic acid sequence encoding the mature light chain variable region of antibody 1415.1.1.

SEQ ID NO:50 is the amino acid sequence of the mature light chain variable region of antibody 1415.1.1.

SEQ ID NO:51 is the amino acid sequence of CDRL1 of antibody 1415.1.1.

SEQ ID NO:52 is the amino acid sequence of CDRL2 of antibody 1415.1.1.

SEQ ID NO:53 is the amino acid sequence of CDRL3 of antibody 1415.1.1.

SEQ ID NO:54 is the nucleic acid sequence encoding the mature heavy chain variable region of antibody 1415.1.1.

SEQ ID NO:55 is the amino acid sequence of the mature heavy chain variable region of antibody 1415.1.1.

SEQ ID NO:56 is the amino acid sequence of CDRH1 of antibody 1415.1.1.

SEQ ID NO:57 is the amino acid sequence of CDRH2 of antibody 1415.1.1.

SEQ ID NO:58 is the amino acid sequence of CDRH3 of antibody 1415.1.1.

SEQ ID NO:59 is the nucleic acid sequence encoding the mature light chain variable region of antibody 1749.1.3.

SEQ ID NO:60 is the amino acid sequence of the mature light chain variable region of antibody 1749.1.3.

SEQ ID NO:61 is the amino acid sequence of CDRL1 of antibody 1749.1.3.

SEQ ID NO:62 is the amino acid sequence of CDRL2 of antibody 1749.1.3.

SEQ ID NO:63 is the amino acid sequence of CDRL3 of antibody 1749.1.3.

SEQ ID NO:64 is the nucleic acid sequence encoding the mature heavy chain variable region of antibody 1749.1.3.

SEQ ID NO:65 is the amino acid sequence of the mature heavy chain variable region of antibody 1749.1.3.

SEQ ID NO:66 is the amino acid sequence of CDRH1 of antibody 1749.1.3.

SEQ ID NO:67 is the amino acid sequence of CDRH2 of antibody 1749.1.3.

SEQ ID NO:68 is the amino acid sequence of CDRH3 of antibody 1749.1.3.

SEQ ID NO:69 is the nucleic acid sequence encoding a mature light chain variable region of antibody 2120.4.19.

SEQ ID NO:70 is the amino acid sequence of the mature light chain variable region of antibody 2120.4.19 set forth in SEQ ID NO:69.

SEQ ID NO:71 is the amino acid sequence of a mature light chain variable region of antibody 2120.4.19.

SEQ ID NO:72 is the amino acid sequence of a mature light chain variable region of antibody 2120.4.19.

SEQ ID NO:73 is the amino acid sequence of CDRL1 of antibody 2120.4.19.

SEQ ID NO:74 is the amino acid sequence of CDRL2 of antibody 2120.4.19.

SEQ ID NO:75 is the amino acid sequence of CDRL3 of antibody 2120.4.19.

SEQ ID NO:76 is the nucleic acid sequence encoding the mature heavy chain variable region of antibody 2120.4.19.

SEQ ID NO:77 is the amino acid sequence of the mature heavy chain variable region of antibody 2120.4.19.

SEQ ID NO:78 is the amino acid sequence of CDRH1 of antibody 2120.4.19.

SEQ ID NO:79 is the amino acid sequence of CDRH2 of antibody 2120.4.19.

SEQ ID NO:80 is the amino acid sequence of CDRH3 of antibody 2120.4.19.

SEQ ID NO:81 is a nucleic acid sequence encoding a mature light chain variable region of antibody 2107.4.10.

SEQ ID NO:82 is the amino acid sequence of the mature light chain variable region of antibody 2107.4.10 set forth in SEQ ID NO:81.

SEQ ID NO:83 is a nucleic acid sequence encoding a mature light chain variable region of antibody 2107.4.10.

SEQ ID NO:84 is the amino acid sequence of the mature light chain variable region of antibody 2107.4.10 set forth in SEQ ID NO:83.

SEQ ID NO:85 is the amino acid sequence of CDRL1 of antibody 2107.4.10.

SEQ ID NO:86 is the amino acid sequence of CDRL2 of antibody 2107.4.10.

SEQ ID NO:87 is the amino acid sequence of CDRL3 of antibody 2107.4.10.

SEQ ID NO:88 is the nucleic acid sequence encoding the mature heavy chain variable region of antibody 2107.4.10.

SEQ ID NO:89 is the amino acid sequence of the mature heavy chain variable region of antibody 2107.4.10.

SEQ ID NO:90 is the amino acid sequence of CDRH1 of antibody 2107.4.10.

SEQ ID NO:91 is the amino acid sequence of CDRH2 of antibody 2107.4.10.

SEQ ID NO:92 is the amino acid sequence of CDRH3 of antibody 2107.4.10.

SEQ ID NO:93 is the amino acid sequence of the mature heavy chain variable region of antibody 1749.1.3.

SEQ ID NO:94 is the amino acid sequence of the mature heavy chain variable region of humanized antibody 1749 version 1 (VH1).

SEQ ID NO:95 is the amino acid sequence of the mature heavy chain variable region of humanized antibody 1749 version 2 (VH2).

SEQ ID NO:96 is the amino acid sequence of the heavy chain variable framework donor U96282_VH.

SEQ ID NO:97 is the amino acid sequence of the mature light chain variable region of antibody 1749.1.3.

SEQ ID NO:98 is the amino acid sequence of the mature light chain variable region of humanized antibody 1749 version 1 (VL1).

SEQ ID NO:99 is the amino acid sequence of the mature light chain variable region of humanized antibody 1749 version 2 (VL2).

SEQ ID NO:100 is the amino acid sequence of the light chain variable framework donor X02990_VL.

SEQ ID NO:101 is the amino acid sequence of the mature heavy chain variable region of antibody 2107.4.10.18.

SEQ ID NO:102 is the amino acid sequence of the mature heavy chain variable region of humanized antibody 2107 version 1 (VH1).

SEQ ID NO:103 is the amino acid sequence of the mature heavy chain variable region of humanized antibody 2107 version 2 (VH2).

SEQ ID NO:104 is the amino acid sequence of the mature heavy chain variable region of humanized antibody 2107 version 3 (VH3).

SEQ ID NO:105 is the amino acid sequence of the mature heavy chain variable region of humanized antibody 2107 version 4A (VH4A).

SEQ ID NO:106 is the amino acid sequence of the mature heavy chain variable region of humanized antibody 2107 version 5A (VH5A).

SEQ ID NO:107 is the amino acid sequence of the mature heavy chain variable region of humanized antibody 2107 version 6 (VH6).

SEQ ID NO:108 is the amino acid sequence of the heavy chain variable framework donor AF062133_VH.

SEQ ID NO:109 is the amino acid sequence of the mature light chain variable region of antibody 2107.4.10.18.

SEQ ID NO:110 is the amino acid sequence of the mature light chain variable region of humanized antibody 2107 version 1 (VL1).

SEQ ID NO:111 is the amino acid sequence of the mature light chain variable region of humanized antibody 2107 version 2 (VL2).

SEQ ID NO:112 is the amino acid sequence of the mature light chain variable region of humanized antibody 2107 version 3 (VL3).

SEQ ID NO:113 is the amino acid sequence of the light chain variable framework donor U86803.

SEQ ID NO:114 is the amino acid sequence of the mature heavy chain variable region of antibody 2120.4.19.6.

SEQ ID NO:115 is the amino acid sequence of the mature heavy chain variable region of humanized antibody 2120 version 1 (VH1).

SEQ ID NO:116 is the amino acid sequence of the mature heavy chain variable region of humanized antibody 2120 version 2 (VH2).

SEQ ID NO:117 is the amino acid sequence of the mature heavy chain variable region of humanized antibody 2120 version 3 (VH3).

SEQ ID NO:118 is the amino acid sequence of the mature heavy chain variable region of humanized antibody 2120 version 4 (VH4).

SEQ ID NO:119 is the amino acid sequence of the mature heavy chain variable region of humanized antibody 2120 version 5 (VH5).

SEQ ID NO:120 is the amino acid sequence of the mature light chain variable region of antibody 2120.4.19.6.

SEQ ID NO:121 is the amino acid sequence of the mature light chain variable region of humanized antibody 2120 version 1 (VL1).

SEQ ID NO:122 is the amino acid sequence of the mature light chain variable region of humanized antibody 2120 version 2 (VL2).

SEQ ID NO:123 is the amino acid sequence of the mature light chain variable region of humanized antibody 2120 version 3 (VL3).

SEQ ID NO:124 is the amino acid sequence of the light chain variable framework donor X84343_VL.

SEQ ID NO:125 is the amino acid sequence of a humanized heavy chain framework region.

SEQ ID NO:126 is the amino acid sequence of a humanized heavy chain framework region.

SEQ ID NO:127 is the amino acid sequence of a humanized heavy chain framework region.

SEQ ID NO:128 is the amino acid sequence of a humanized heavy chain/light chain framework region.

SEQ ID NO:129 is the amino acid sequence of a humanized light chain framework region.

SEQ ID NO:130 is the amino acid sequence of a humanized light chain framework region.

SEQ ID NO:131 is the amino acid sequence of a humanized light chain framework region.

SEQ ID NO:132 is the amino acid sequence of a humanized light chain framework region.

SEQ ID NO:133 is the amino acid sequence of a humanized heavy chain framework region.

SEQ ID NO:134 is the amino acid sequence of a humanized heavy chain framework region.

SEQ ID NO:135 is the amino acid sequence of a humanized heavy chain framework region.

SEQ ID NO:136 is the amino acid sequence of a humanized heavy chain framework region.

SEQ ID NO:137 is the amino acid sequence of a humanized heavy chain framework region.

SEQ ID NO:138 is the amino acid sequence of a humanized heavy chain framework region.

SEQ ID NO:139 is the amino acid sequence of CDRH1 of humanized antibody 2120 version 3 (VH3).

SEQ ID NO:140 is the amino acid sequence of CDRH1 of humanized antibody 2120 version 4 (VH4).

SEQ ID NO:141 is the amino acid sequence of CDRH1 of humanized antibody 2120 version 5 (VH5).

SEQ ID NO:142 is the amino acid sequence of a humanized light chain framework region.

SEQ ID NO:143 is the amino acid sequence of a humanized light chain framework region.

EQ ID NO:144 is the amino acid sequence of a humanized light chain framework region.

SEQ ID NO:145 is the amino acid sequence of a humanized light chain framework region.

SEQ ID NO:146 is the amino acid sequence of a humanized light chain framework region.

SEQ ID NO:147 is the amino acid sequence of a humanized light chain framework region.

SEQ ID NO:148 is the amino acid sequence of a humanized light chain framework region.

SEQ ID NO:149 is the amino acid sequence of a humanized light chain framework region.

SEQ ID NO:150 is the amino acid sequence of a humanized light chain framework region.

SEQ ID NO:151 is the amino acid sequence of CDRH1 of humanized antibody 2107 version 1 (VH1).

SEQ ID NO:152 is the amino acid sequence of CDRH1 of humanized antibody 2107 version 4 (VH4).

SEQ ID NO:153 is the amino acid sequence of CDRH3 of humanized antibody 2120 version 1-5 (VH1-VH5).

SEQ ID NO:154 is the amino acid sequence of a humanized light chain framework region.

SEQ ID NO:155 is the amino acid sequence of a humanized heavy chain framework region.

SEQ ID NO:156 is the amino acid sequence of the mature heavy chain variable region of antibody 2120.4.19.Q1E, wherein position 1 (Kabat numbering) is occupied by E.

SEQ ID NO:157 is the amino acid sequence of the mature heavy chain variable region of humanized antibody 2120 version 1 Q1E (VH1.Q1E), wherein position 1 (Kabat numbering) is occupied by E.

SEQ ID NO:158 is the amino acid sequence of the mature heavy chain variable region of humanized antibody 2120 version 2 Q1E (VH2.Q1E), wherein position 1 (Kabat numbering) is occupied by E.

SEQ ID NO:159 is the amino acid sequence of the mature heavy chain variable region of humanized antibody 2120 version 3 Q1E (VH3.Q1E), wherein position 1 (Kabat numbering) is occupied by E.

SEQ ID NO:160 is the amino acid sequence of the mature heavy chain variable region of humanized antibody 2120 version 4 Q1E (VH4.Q1E), wherein position 1 (Kabat numbering) is occupied by E.

SEQ ID NO:161 is the amino acid sequence of the mature heavy chain variable region of humanized antibody 2120 version 5 Q1E (VH5.Q1E), wherein position 1 (Kabat numbering) is occupied by E.

SEQ ID NO:162 is the nucleic acid sequence encoding an exemplary signal peptide that can be fused to a mature heavy chain or mature light chain variable region.

SEQ ID NO:163 is the amino acid sequence of the exemplary signal peptide encoded by the nucleic acid sequence of SEQ ID NO:162.

SEQ ID NO:164 is the nucleic acid sequence encoding an exemplary signal peptide that can be fused to a mature heavy chain or mature light chain variable region.

SEQ ID NO:165 is the amino acid sequence of the exemplary signal peptide encoded by the nucleic acid sequence of SEQ ID NO:164.

SEQ ID NO:166 is the nucleic acid sequence encoding an exemplary signal peptide that can be fused to a mature heavy chain or mature light chain variable region.

SEQ ID NO:167 is the amino acid sequence of the exemplary signal peptide encoded by the nucleic acid sequence of SEQ ID NO:166.

SEQ ID NO:168 is the amino acid sequence of a humanized 2120 light chain constant region, with Arginine at the N-terminus.

SEQ ID NO:169 is the amino acid sequence of a humanized 2120 light chain constant region, without Arginine at the N-terminus.

SEQ ID NO:170 is the amino acid sequence of a humanized 2120 heavy chain constant region.

SEQ ID NO:171 is the amino acid sequence of a BIP version heavy chain G1m3 allotype constant region.

SEQ ID NO:172 is the amino acid sequence of a BIP version heavy chain G1m3 allotype constant region.

SEQ ID NO:173 is the amino acid sequence of a mature light chain region of humanized antibody 2120 version 3 (VL3+light chain constant region).

SEQ ID NO:174 is the amino acid sequence of a mature heavy chain region of humanized antibody 2120 version 5 (VH5+BIP version heavy chain G1m3 allotype constant region).

SEQ ID NO:175 is the amino acid sequence of a mature heavy chain region of humanized antibody 2120 version 5 (VH5+BIP version heavy chain G1m3 allotype constant region).

SEQ ID NO:176 is the amino acid sequence of a mature heavy chain region of humanized antibody 2120 version 5 Q1E (VH5.Q1E+BIP version heavy chain G1m3 allotype constant region). SEQ ID NO:177 is the amino acid sequence of a mature heavy chain region of humanized antibody 2120 version 5 Q1E (VH5.Q1E+BIP version heavy chain G1m3 allotype constant region).

SEQ ID NO:178 is the amino acid sequence of the mature heavy chain variable region of humanized antibody 2107 version 4B (VH4B).

SEQ ID NO:179 is the amino acid sequence of the mature heavy chain variable region of humanized antibody 2107 version 5B (VH5B).

### DEFINITIONS

Monoclonal antibodies are typically provided in isolated form. This means that an antibody is typically at least 50% w/w pure of proteins and other macromolecules arising from its production or purification but does not exclude the possibility that the monoclonal antibody is combined with an excess of pharmaceutical acceptable carrier(s) or other vehicle intended to facilitate its use. Sometimes monoclonal antibodies are at least 60%, 70%, 80%, 90%, 95 or 99% w/w pure of proteins and other macromolecules from production or purification.

Specific binding of a monoclonal antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a monoclonal antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989, Ch. 7, incorporated by reference in its entirety for all purposes).

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chains or between different light chains are assigned the same number (e.g., H83 means position 83 by Kabat numbering in the mature heavy chain variable region; likewise position L36 means position 36 by Kabat numbering in the mature light chain variable region). Kabat numbering is used throughout in referring to positions in the variable region of an antibody unless explicitly stated otherwise.

The term “antibody” includes intact antibodies and antigen binding fragments thereof. Typically, fragments compete with the intact antibody from which they were derived for specific binding to the target including separate heavy chains, light chains Fab, Fab′, F(ab′)₂, F(ab)c, diabodies, Dabs, nanobodies, and Fv. Fragments can be produced by recombinant DNA techniques, or by enzymatic or chemical separation of intact immunoglobulins.

The term “antibody” also includes a bispecific antibody, and/or a chimeric antibody, and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol. 148:1547-53 (1992)). In some bispecific antibodies, the two different heavy/light chain pairs may include a humanized heavy chain/light chain pair and a heavy chain/light chain pair specific for a different epitope.

In some bispecific antibodies, one heavy chain light chain pair is a humanized antibody as further disclosed below and the heavy light chain pair is from an antibody that binds to a receptor expressed on the blood brain barrier, such as an insulin receptor, an insulin-like growth factor (IGF) receptor, a leptin receptor, or a lipoprotein receptor, or a transferrin receptor (Friden et al., PNAS 88:4771-4775, 1991; Friden et al., Science 259:373-377, 1993). Such a bispecific antibody can be transferred cross the blood brain barrier by receptor-mediated transcytosis. Brain uptake of the bispecific antibody can be further enhanced by engineering the bi-specific antibody to reduce its affinity to the blood brain barrier receptor. Reduced affinity for the receptor resulted in a broader distributioin in the brain (see, e.g., Atwal. et al. Sci. Trans. Med. 3, 84ra43, 2011; Yu et al. Sci. Trans. Med. 3, 84ra44, 2011).

Exemplary bispecific antibodies can also be (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (5) a so-called Scorpion molecule, comprising, e.g., two scFvs fused to both termini of a human Fc-region. Examples of platforms useful for preparing bispecific antibodies include but are not limited to BiTE (Micromet), DART (MacroGenics), Fcab and Mab2 (F-star), Fc-engineered IgGl (Xencor) or DuoBody (based on Fab arm exchange, Genmab).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

An “antagonist” antibody or other binding agent is one which inhibits a biological activity of the antigen it binds. Such antibodies may substantially or completely inhibit the biological activity of the antigen.

The terms “biological activity” and “biologically active” with regard to MCAM refer to its ability to specifically bind its ligand (a laminin α4 chain, e.g., the α4 chain of laminin 411) and/or to facilitate the infiltration of MCAM-expressing cells, e.g., TH17 cells, into the CNS.

“Inhibit” means an agent decreases the biological activity of at least one target, for example MCAM. Such an inhibitor inhibits the activity of at least one target by at least about at least 25%, at least 30%, at least 35%, at least 40%, at least 45%, at least 50%, at least 55%, at least 60%, at least 65%, at least 70%, at least 75%, at least 80%, at least 85%, at least 95% or at least 100%.

A “subject” includes a human or other mammalian subject that receives either prophylactic or therapeutic treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Nonconservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Designation of a range of values includes all integers within or defining the range, and all subranges defined by integers within the range.

Unless otherwise apparent from the context, the term “about” encompasses values within a standard margin of error of measurement (SEM) of a stated value.

Statistical significance means p≦0.05.

### DETAILED DESCRIPTION

### I. General

Antibodies with the useful property of inhibiting MCAM binding to the laminin α4 chain of laminin 411 are disclosed in WO/2012/170071 and PCT/US2013/058773. U.S. Ser. No. 14/656,596 provides among other things (a) provides new humanized forms of the 2120.4.19 antibody, (b) maps the epitope to which this antibody binds, (c) provides antibodies binding to the same epitope, and (d) provides a new formulation of the disclosed antibodies. The present application discloses the use of the antibodies and formulations of Ser. No. 14/656,596 for treatment or prophylaxis of giant cell arteritis, polymyalgia rheumatica (PMR) or Takayasu's arteritis,

The terms “2120.4.19”, “m2120”, “mouse 2120” antibody refer to a rodent derived monoclonal antibody clone having a mature variable heavy chain corresponding to SEQ ID NO:114 and a mature variable light chain corresponding to SEQ ID NO:120. “Humanized 2120” or “hu2120” refers to humanized variants of the 2120.4.19 clone.

### II. Target Molecules

Natural human wild-type MCAM (melanoma cell adhesion molecule, also known as CD146 and MUC18) is a protein of 646 amino acids having the following amino acid sequence:

(SEQ ID NO: 11)
MGLPRLVCAFLLAACCCCPRVAGVPGEAEQPAPELVEVEVGSTALLKCGL
SQSQGNLSHVDWFSVHKEKRTLIFRVRQGQGQSEPGEYEQRLSLQDRGAT
LALTQVTPQDERIFLCQGKRPRSQEYRIQLRVYKAPEEPNIQVNPLGIPV
NSKEPEEVATCVGRNGYPIPQVIWYKNGRPLKEEKNRVHIQSSQTVESSG
LYTLQSILKAQLVKEDKDAQFYCELNYRLPSGNHMKESREVTVPVFYPTE
KVWLEVEPVGMLKEGDRVEIRCLADGNPPPHFSISKQNPSTREAEEETTN
DNGVLVLEPARKEHSGRYECQAWNLDTMISLLSEPQELLVNYVSDVRVSP
AAPERQEGSSLTLTCEAESSQDLEFQWLREETDQVLERGPVLQLHDLKRE
AGGGYRCVASVPSIPGLNRTQLVKLAIFGPPWMAFKERKVWVKENMVLNL
SCEASGHPRPTISWNVNGTASEQDQDPQRVLSTLNVLVTPELLETGVECT
ASNDLGKNTSILFLELVNLTTLTPDSNTTTGLSTSTASPHTRANSTSTER
KLPEPESRGVVIVAVIVCILVLAVLGAVLYFLYKKGKLPCRRSGKQEITL
PPSRKTELVVEVKSDKLPEEMGLLQGSSGDKRAPGDQGEKYIDLRH.

(GenBank database under Accession Number AAA20922.1 (CAA48332)). MCAM is a cell surface glycoprotein belonging to the immunoglobulin superfamily involved in cell adhesion, and in cohesion of the endothelial monolayer at intercellular junctions in vascular tissue. It also promotes tumor progression of many cancers, such as solid tumors, including melanoma and prostate cancer. It is known to interact in a homotypic/homophilic manner and may also bind to other ligands. The human MCAM includes five immunoglobulin domains (1: amino acid residues 19-129; 2: amino acid residues 139-242; 3: amino acid residues 244-321; 4: amino acid residues 335-424; and 5: amino acid residues 430-510), shown as SEQ ID NOS:22-26.

Unless otherwise apparent from the context, reference to MCAM or its fragments includes the natural human wildtype amino acid sequences indicated above, and human allelic variants thereof.

Laminin α4 refers to one of the polypeptide chains found in laminin molecules, which are expressed in the basal lamina (of the basement membrane), a protein network foundation for most cells and organs. Laminins are known to bind to cell membranes through plasma membrane molecules and contribute to cell attachment. The laminin α4 chain typically forms a complex with a laminin β-chain, and a laminin γ-chain. The laminin α4 chain is found in numerous laminin molecules including laminin 411 (laminin 8 or α4β1γ1); laminin 421 (laminin 9 or α4β2γ1), and laminin 423 (laminin 14 or α4β2γ3). There are two main isoforms of the human laminin α4-chain: GenBank Accession Nos. NP001098676 and NP001098677 (SEQ ID NOS:27 and 28, respectively). “Laminin 411” refers to a trimeric polypeptide complex made up of three polypeptide subunits or chains: α4-chain, a β1-chain, and a γ1-chain.

Antagonist against MCAM include antibodies, fusion proteins of receptors or ligands to an IgG constant region other biologic binding molecules, and small molecules. Antibodies can be monoclonal or polyclonal. Antibodies can be nonhuman, such as mouse or rat, nonhuman primate or can be human. Antibodies can be chimeric, veneered, humanized, primatized and the like.

An MCAM antagonist refers to an antagonist that fully or partially inhibits the ability of MCAM (i) to specifically bind its ligand: a laminin α4 chain, e.g., the α4 chain of laminin 411; and/or (ii) to facilitate an MCAM-expressing cell, e.g., a TH17 cell, to infiltrate into or migrate to a subject's tissue. MCAM antagonists include antibodies or other antagonists binding to MCAM or to its ligand laminin alpha 4.

### III. Antibodies

### A. Antibody 2120.4.19 and Chimeric, Veneered, and Humanized Forms Thereof

A humanized antibody is a genetically engineered antibody in which the CDRs from a non-human “donor” antibody (i.e., 2120.4.19) are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs, except CDRH1 can have up to two substitutions and CHDRH2 can have substitutions at positions H60-65. The variable region framework sequences of an antibody chain or the constant region of an antibody chain are substantially from a human variable region framework sequence or human constant region respectively when at least 85%, 90%, 95% or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs) from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The 2120.4.19 rat antibody against MCAM was disclosed in PCT/US2013/058773 and is defined herein by SEQ ID NOs:69-80. Chimeric, veneered, and humanized forms of the 2120.4.19 antibody were also disclosed in the '773 application. The disclosed humanized forms are defined herein as SEQ ID NOs:115-119, 121-123, 139-141, and 153. The disclosed forms including any permutation of a humanized heavy chain and humanized light chain represented by these SEQ ID NOS. can be used in some aspects of the present invention, such as pharmaceutical compositions and formulations.

The present application provides additional humanized forms of the 2120.4.19 antibody in which glutamine is substituted to glutamic acid at position 1 (Kabat numbering) of the heavy chain variable region (i.e. Q1E). The Q1E substitution in the heavy chain variable region is a conservative substitution not expected to produce a substantial effect on the binding characteristics of the antibody, but which can improve antibody stability.

Unless otherwise apparent from the context, the following description includes the humanized antibodies disclosed in PCT/US2013/058773 and the Q1E variants disclosed herein.

The disclosure provides antibodies comprising a heavy chain variable region comprising Kabat CDR1 of SEQ ID NO:78: GFSLTSNGVS; Kabat CDR2 of SEQ ID NO:79: AISSGGTTYYNSAFKS; and Kabat CDR3 of SEQ ID NO:80: RYGYGWYFDF. Some antibodies comprise a light chain variable region comprising Kabat CDR1 of SEQ ID NO:73: KASQNIYNSLA; Kabat CDR2 of SEQ ID NO:74: NANSLQT; and Kabat CDR3 of SEQ ID NO:75: QQFYSGYT. Some such antibodies comprise an N32S substitution or an N32Q substitution in Kabat CDR1 of SEQ ID NO:78, and some comprise a G33A substitution in Kabat CDR1 of SEQ ID NO:78. These substitutions have been found to offer improved characteristics including an increase in antibody affinity and potency.

The disclosure also provides anti-MCAM antibodies in which the mature heavy chain variable region has at least 90%, 95%, 96%, 97%, 98% or 99% identity to SEQ ID NO:161, and the mature light chain variable region has at least 90%, 95%, 96%, 97%, 98% or 99% sequence identity to SEQ ID NO:123. Some such antibodies include three heavy chain and three light chain CDRs entirely or substantially identical to the CDR regions of the donor 2120.4.19 antibody. If not identical, CDRs preferably have substitutions at a type and position defined herein, such as in the previous paragraph. The CDR regions can be defined by any conventional definition (e.g., Chothia) but are preferably as defined by Kabat.

Any of the above described antibodies can be humanized antibodies. Some humanized antibodies comprise a mature heavy chain variable region comprising the three Kabat CDRs of SEQ ID NO:161 (which are the same as the CDRs of SEQ ID NO:156) except that position 32 (Kabat numbering) can be N, S, or Q, and position 33 (Kabat numbering) can be G or A, and a mature light chain variable region comprising the three Kabat CDRs of SEQ ID NO:123 (which are the same as the CDRs of SEQ ID NO:120), preferably wherein the mature heavy chain variable region is at least 90% identical to SEQ ID NO:161, and preferably wherein the mature light chain variable region is at least 90% identical to SEQ ID NO:123. Any such antibody can have either Q or E (i.e., Q1E substitution) at position H1 by Kabat numbering.

The antibodies provided herein having a Q1E substitution in the mature heavy chain variable region include antibodies comprising a mature heavy chain variable region having the amino acid sequence of SEQ ID NO:156 (i.e., 2120.4.19.Q1E), SEQ ID NO:157, SEQ ID NO:158, SEQ ID NO:159, SEQ ID NO:160, or SEQ ID NO:161. Some such antibodies comprise a mature light chain variable region having the amino acid sequence designated SEQ ID NO:120, SEQ ID NO:121, SEQ ID NO:122, or SEQ ID NO:123. The mature heavy chain and light chain variable regions can be combined in any possible permutation. An exemplary combination is an antibody that comprises the mature heavy chain variable region having the amino acid sequence of SEQ ID NO:161, and the mature light chain variable region having the amino acid sequence designated SEQ ID NO:123. Forms of these antibodies without the Q1E substitution, such as have been described in PCT/US2013/058773, can also be used in some aspects of the invention, such as pharmaceutical compositions and formulations.

The disclosure further provides antibodies in which the heavy chain mature variable region has at least 90%, 95%, 96%, 97%, 98%, 99% or 100% sequence identity to the amino acid sequence of any of SEQ ID NO:156 (i.e., 2120.4.19.Q1E), SEQ ID NO:157, SEQ ID NO:158, SEQ ID NO:159, SEQ ID NO:160, or SEQ ID NO:161 and the light chain has at least 90%, 95%, 96%, 97%, 98% or 99% sequence identity to any of SEQ ID NO:120, SEQ ID NO:121, SEQ ID NO:122, or SEQ ID NO:123. Such antibodies are preferably humanized. Any such antibody can have either Q or E (i.e., Q1E substitution) at position H1 by Kabat numbering.

Variants of disclosed SEQ ID NOs typically differ from the mature heavy chain and light chain variable region sequences by a small number (e.g., typically no more than 1, 2, 3, 5 or 10 in either the light chain or heavy chain mature variable region framework, or both) of replacements, deletions or insertions. Any changes are preferably conservative substitutions.

The disclosure also provides humanized forms of 2107 for use in treatment or prophylaxis of Giant Cell Arteritis, Polymyalgia Rheumatica or Takayasu's Arteritis. Such antibodies can include any of the 2107 heavy and light chain variable regions described herein. The heavy chain variable region can be for example that having the sequence of SEQ ID NO:178 or 179. The light chain variable region can be for example that having the sequence of SEQ ID NO:98 or SEQ ID NO:99.

### B. Selection of Constant Region

The heavy and light chain variable regions of chimeric, veneered or humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have complement-dependent cytotoxicity and human isotypes IgG2 and IgG4 do not. Human IgG1 and IgG3 also induce stronger cell mediated effector functions than human IgG2 and IgG4. Light chain constant regions can be lambda or kappa.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduce affinity for Fcγ receptors, particularly FcγRI receptor (see, e.g., U.S. Pat. No. 6,624,821). An alanine substitution at positions 234, 235, and 237 of human IgG1 can be used for reducing effector functions. Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821). In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine, such as in SEQ ID NO:172. In some antibodies, the isotype is human IgG2 or IgG4. An exemplary human light chain kappa constant region has the amino acid sequence of SEQ ID NO:168. The N-terminal arginine of SEQ ID NO:168 can be omitted, in which case light chain kappa constant region has the amino acid sequence of SEQ ID NO:169. An exemplary human IgG1 heavy chain constant region has the amino acid sequence of SEQ ID NO:170 (with or without the C-terminal lysine). Antibodies can be expressed as tetramers containing two light and two heavy chains, as separate heavy chains, light chains, as Fab, Fab′, F(ab′)2, and Fv, or as single chain antibodies in which heavy and light chain mature variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Thus, for example, another heavy chain constant region is of IgG1 G1m3 allotype and has the amino acid sequence of SEQ ID NO:171. Another heavy chain constant region has the amino acid sequence of SEQ ID NO:171 except that it lacks the C-terminal lysine. Another heavy chain constant region has the amino acid sequence of SEQ ID NO:172. Yet another heavy chain constant region has the amino acid sequence of SEQ ID NO:172 except that it lacks the C-terminal lysine.

The disclosure further provides nucleic acids encoding any of the above constant regions. Optionally, such nucleic acids further encode a signal peptide and can be expressed with the signal peptide linked to the constant region.

### C. Expression of Recombinant Antibodies

Antibodies can be produced by recombinant expression. Nucleic acids encoding the antibodies can be codon-optimized for expression in the desired cell-type (e.g., CHO or Sp2/0). Recombinant nucleic acid constructs typically include an expression control sequence operably linked to the coding sequences of antibody chains, including naturally-associated or heterologous promoter regions. The expression control sequences can be eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies. The vector or vectors encoding the antibody chains can also contain a selectable gene, such as dihydrofolate reductase, to allow amplification of copy number of the nucleic acids encoding the antibody chains.

E. coli is a prokaryotic host particularly useful for expressing antibodies, particularly antibody fragments. Microbes, such as yeast are also useful for expression. Saccharomyces is an example of a yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilizations. Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, various COS cell lines, HeLa cells, HEK293 cells, L cells, and non-antibody-producing myelomas including Sp2/0 and NS0. It can be advantageous to use nonhuman cells. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Suitable expression control sequences are promoters derived from endogenous genes, cytomegalovirus, SV40, adenovirus, bovine papillomavirus, and the like. See Co et al., J. Immunol. 148:1149 (1992).

Having introduced vector(s) encoding antibody heavy and light chains into cell culture, cell pools can be screened for growth productivity and product quality in serum-free media. Top-producing cell pools can then be subjected to FACS-based single-cell cloning to generate monoclonal lines. Specific productivities above 50 pg or 100 pg per cell per day, which correspond to product titers of greater than 7.5 g/L culture, can be advantageous. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, antibodies can be purified according to standard procedures of the art, including protein A capture, column chromatography (e.g., hydrophobic interaction or ion exchange), low-pH for viral inactivation and the like (see generally, Scopes, Protein Purification (Springer-Verlag, NY, 1982)).

Methodology for commercial production of antibodies including codon optimization, selection of promoters, transcription elements, and terminators, serum-free single cell cloning, cell banking, use of selection markers for amplification of copy number, CHO terminator, serum free single cell cloning, improvement of protein titers (see, e.g., U.S. Pat. No. 5,786,464, U.S. Pat. No. 5,888,809, U.S. Pat. No. 6,063,598, U.S. Pat. No. 6,114,148, U.S. Pat. No. 7,569,339, WO2004/050884, WO2005/019442, WO2008/012142, WO2008/012142, WO2008/107388, and WO2009/027471).

### D. Nucleic Acids

The disclosure further provides nucleic acids encoding any of the heavy and light chains described above. Typically, the nucleic acids also encode a signal peptide fused to the mature heavy and light chains (e.g., signal peptides having amino acid sequences of SEQ ID NOs:163, 165, and 167 that can be encoded by SEQ ID NOS:162, 164, and 166). Coding sequences on nucleic acids can be in operable linkage with regulatory sequences to ensure expression of the coding sequences, such as a promoter, enhancer, ribosome binding site, transcription termination signal and the like. The nucleic acids encoding heavy and light chains can occur in isolated form or can be cloned into one or more vectors. The nucleic acids can be synthesized by for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

E. Characterization of MCAM Epitopes for Antibody Binding and Production of Antibodies that Bind the Same

### 1. MCAM Epitopes for Antibody Binding

The disclosure provides monoclonal antibodies that bind to specific epitopes within the human MCAM protein, some of which bind to the same or overlapping epitope as the antibody designated 2120.4.19 (m2120).

Mutations at residues 39, 62, 133, 141, 159, 212, 220, 221, 223, 227, 238, 241, and/or 392 of MCAM (SEQ ID NO:11) disrupt specific binding of m2120 (e.g., <30% binding to mutant MCAM compared to a positive control wild type MCAM as described as the examples). Mutations at residues 145, 167, 175, 206, 207, 216, and 225 were identified as having the greatest effect (reduction) of specific binding of m2120. Because relatively few residues affect binding and the residues are spaced more broadly than a typical linear epitope (e.g., 3-20 contiguous amino acids), these results provide an indication that m2120 may bind to a conformational epitope or, alternatively, one or more of the residues affecting binding may do so allosterically without direct contact with the antibody.

Antibodies binding to an epitope including one or more of residues 39, 62, 133, 141, 145, 159, 167, 175, 206, 207, 212, 216, 220, 221, 223, 225, 227, 238, 241, and 392 of MCAM, and particularly to an epitope including one or more of residues 141 and 145, are likely to share useful inhibitory properties with m2120. Thus, antibodies whose specific binding is inhibited by mutagenesis of one or more or residues 141 and 145 and particularly residue 141 of MCAM are likely to share similar properties to m2120. Some such antibodies bind to an epitope that includes or consists of residue 141 and/or 145 of MCAM. The epitope can be linear, such as an epitope (e.g., 2-5, 3-5, 3-10, 3-15, 3-20, 5-10, 5-15, 5-20, 5-30, 5-40, 5-50, 5-60, or 5-70 contiguous amino acids) including one or both of the specified amino acids (141 and 145) or can be conformational including or consisting of 1 or both of the specified amino acids.

2. The Generation of Antibodies that Bind Specific MCAM Epitopes

Some antibodies bind to the same or overlapping epitope as the m2120 antibody. The production of other non-human monoclonal antibodies, e.g., murine, guinea pig, primate, rabbit or rat, against human MCAM can be accomplished by, for example, immunizing the animal with human MCAM or a peptide fragment or a cell line displaying human MCAM or human MCAM cDNA (encoding by retrovirus or immunizing with a gene gun) thereof including the desired epitope (the “immunogen”), and screening resulting antibodies for binding to MCAM, optionally in competition with m2120 (See Harlow & Lane, Antibodies, A Laboratory Manual (CSHP NY, 1988) incorporated by reference for all purposes). Optionally, the immunogen is conjugated to carrier molecule. Optionally, the immunogen is administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to a desired epitope within MCAM.

The disclosure provides peptide fragments of MCAM that are used to create antibodies directed to the above described epitopes. Examples of such peptides include a peptide that is between 2-5, 3-5, 3-10, 3-15, 3-20, 5-10, 5-15, 5-20, 5-30, 5-40, 5-50, 5-60, or 5-70 contiguous amino acids in length and includes at least one of amino acids residue 141 and 145 of MCAM. In some of these peptides, the peptide includes both of amino acid residues 141 and 145.

Immunogens may be conjugated to carrier molecules, typically a carrier polypeptide, and thus help elicit an immune response against the fragment conjugated to the carrier. A single agent can be linked to a single carrier, multiple copies of an agent can be linked to multiple copies of a carrier, which are in turn linked to each other, multiple copies of an agent can be linked to a single copy of a carrier, or a single copy of an agent can be linked to multiple copies of a carrier, or different carriers. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM₁₉₇), E. coli, cholera, or H. pylori, or an attenuated toxin derivative.

Immunogens are often administered with pharmaceutically acceptable adjuvants. The adjuvant increases the titer of induced antibodies and/or the binding affinity of induced antibodies relative to the situation if the peptide were used alone. A variety of adjuvants can be used in combination with an immunogenic fragment of MCAM, to elicit an immune response. Preferred adjuvants augment the intrinsic response to an immunogen without causing conformational changes in the immunogen that affect the qualitative form of the response. Exemplary adjuvants include aluminum hydroxide and aluminum phosphate, 3 De-O-acylated monophosphoryl lipid A (MPL™) (see GB 2220211 (RIBI ImmunoChem Research Inc., Hamilton, Mont., now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, Mass.; now Antigenics, Inc., New York, N.Y.). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

### 3. Types of Antibodies

Antibodies can be monoclonal or polyclonal. Antibodies can be nonhuman, such as mouse or rat, nonhuman primate or can be human. Antibodies can be chimeric, veneered, humanized, primatized and the like.

Monoclonal antibodies are humanized using the methods described above and the methods described in Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557. The acceptor antibody sequences can be, for example, a mature human antibody variable region sequence, a composite of such sequences, a consensus sequence of human antibody variable region sequences (e.g., light and heavy chain variable region consensus sequences of Kabat, 1991, supra), or a germline variable region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:

(1) noncovalently binds antigen directly,

(2) is adjacent to a CDR region,

(3) otherwise interacts with a CDR region (e.g. is within about 6 Å of a CDR region).

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position.

The disclosure further provides chimeric and veneered forms of non-human antibodies that bind specifically to the MCAM epitopes described above.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues with residues from the corresponding positions of a human antibody sequence (Padlan, Mol. Immunol. 28:489, 1991). The result is an antibody in which the CDRs are entirely or substantially from a non-human antibody and the variable region frameworks of the non-human antibody are made more human-like by the substitutions.

Human antibodies against MCAM are provided by a variety of techniques described below. Some human antibodies are selected by competitive binding experiments, by the phage display method of Winter, above, or otherwise, to have the same epitope specificity as a particular mouse antibody, such as one of the mouse monoclonals described in the examples. Human antibodies can also be screened for a particular epitope specificity by using only a fragment of MCAM as the target antigen, and/or by screening antibodies against a collection of deletion mutants of MCAM.

Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332.

Chimeric, humanized (including veneered) and human antibodies are typically produced by recombinant expression as described above.

The disclosure further provides non-antibody binding molecules. Non-antibody binding molecules include, for example, anticalins, which are based upon the lipocalin scaffold, a protein structure characterized by a rigid beta-barrel that supports four hypervariable loops which form the ligand binding site. Novel binding specificities are engineered by targeted random mutagenesis in the loop regions, in combination with functional display and guided selection (Skerra (2008) FEBS J. 275: 2677-2683). Other suitable scaffolds may include, for example, adnectins, or monobodies, based on the tenth extracellular domain of human fibronectin III (Koide and Koide (2007) Methods Mol. Biol. 352: 95-109); affibodies, based on the Z domain of staphylococcal protein A (Nygren et al. (2008) FEBS J. 275: 2668-2676)); DARPins, based on ankyrin repeat proteins (Stumpp et al. (2008) Drug. Discov. Today 13: 695-701); fynomers, based on the SH3 domain of the human Fyn protein kinase (Grabulovski et al. (2007) J. Biol. Chem. 282: 3196-3204); affitins, based on Sac7d from Sulfolobus acidolarius (Krehenbrink et al. (2008) J. Mol. Biol. 383: 1058-1068); affilins, based on human y-B-crystallin (Ebersbach et al. (2007) J. Mol. Biol. 372: 172-185); avimers, based on the A domains of membrane receptor proteins (Silverman et al. (2005) Biotechnol. 23: 1556-1561); cysteine-rich knottin peptides (Kolmar (2008) FEBS J. 275: 2684-2690); and engineered Kunitz-type inhibitors (Nixon and Wood (2006) Curr. Opin. Drug. Discov. Dev. 9: 261-268). For review, see Gebauer and Skerra (2009) Curr. Opin. Chem. Biol. 13: 245-255.

In some of these antibodies, the antibody is not any one of the antibodies or antibodies including CDRs (as defined by Kabat, Chothia or a composite thereof) entirely or substantially from the antibodies described in WO/2012/170071 and PCT/US2013/058773, particularly the antibodies designated clone 15 (defined by SEQ ID NOs:12-21) and clone 17 (defined by SEQ ID NOs:1-10) in WO/2012/170071 and the mouse anti-human MCAM monoclonal clones designated 1174.1.3, 1414.1.2, 1415.1.1, and 1749.1.3, and the rat anti-human MCAM monoclonal antibody clones designated 2120.4.19 and 2107.4.10 described in PCT/US2013/058773.

### 4. Methods of Screening Antibodies for Activity

The inhibitory activity of the MCAM antibodies described herein can be assayed by various methods including competitive binding assays with antibodies that bind the same or a substantially similar epitope (e.g., m2120) and blocking of MCAM binding with its ligand, the laminin α4 chain of laminin 411.

For example, the activity of MCAM antibodies to inhibit the interaction between MCAM and the laminin α4 chain of laminin 411 can be screened as follows. MCAM-expressing cells are (a) incubating with a recombinant polypeptide comprising a laminin α4 chain, e.g., an α4 chain of laminin 411, in the presence or absence of a candidate antibody; (b) monitoring the level of binding of the laminin α4 to the cells, e.g. by fluorescence microscopy or flow cytometry; and (c) identifying said candidate antibody as an inhibitor the MCAM/laminin α4 interaction if the level of laminin α4 binding is lower in the presence than in the absence of the candidate antibody. An alternate screening protocol involves the use of a population of cells expressing a laminin α4 chain, which can be incubated with MCAM, in the presence and absence of a candidate antibody, and binding of MCAM to the cell population monitored. If the binding of MCAM to the cell population in the presence of the candidate antibody is lower than in its absence, the candidate antibody is an MCAM antagonist.

Other methods of monitoring include fluorescence-activated cell sorting (FACS) and enzyme-linked immunosorbent assay (ELISA).

The MCAM antagonists identified based on their ability to inhibit the binding of MCAM to its ligand, e.g., a laminin α4 chain, are candidates for the treatment of inflammatory conditions characterized by infiltration of MCAM-expressing cells.

### F. Conjugated Antibodies

Conjugated antibodies that specifically bind to MCAM can be useful in targeting cancer or tumor cells for destruction or in targeting cells involved in autoimmune diseases or neuroinflammatory diseases. Such antibodies can also be useful in targeting any disease mediated at least in part by expression of MCAM. For example, such antibodies can be conjugated with other therapeutic agents, other proteins, other antibodies, and/or detectable labels. See WO 03/057838; U.S. Pat. No. 8,455,622. Such therapeutic agents can be any agent that can be used to treat, combat, ameliorate, prevent, or improve an unwanted condition or disease in a patient, such as an autoimmune disease, a neuroinflammatory disease, or a cancer. Therapeutic agents can include cytotoxic agents, cytostatic agents, radiotherapeutic agents, immunomodulators, or any biologically active agents that facilitate or enhance the activity of the antibody. A cytotoxic agent can be any agent that is toxic to a cell. A cytostatic agent can be any agent that inhibits cell proliferation. An immunomodulator can be any agent that stimulates or inhibits the development or maintenance of an immunologic response. A radiotherapeutic agent can be any molecule or compound that emits radiation. If such therapeutic agents are coupled to an MCAM-specific antibody, such as the antibodies described herein, the coupled therapeutic agents will have a specific affinity for MCAM-expressing cells (e.g., immune cells, such as TH17-expressing cells, or cancer cells, such as malignant melanocytes) over other cells. Consequently, administration of the conjugated antibodies directly targets MCAM-expressing cells with minimal effects on other surrounding cells and tissue. This can be particularly useful for therapeutic agents that are too toxic to be administered on their own. In addition, smaller quantities of the therapeutic agents can be used.

Antibodies can be modified to act as immunotoxins. See, e.g., U.S. Pat. No. 5,194,594. For example, ricin, a cellular toxin derived from plants, can be coupled to antibodies by using the bifunctional reagents S-acetylmercaptosuccinic anhydride for the antibody and succinimidyl 3-(2-pyridyldithio)propionate for ricin. See Pietersz et al., Cancer Res. 48(16):4469-4476 (1998). The coupling results in loss of B-chain binding activity of ricin, while impairing neither the toxic potential of the A-chain of ricin nor the activity of the antibody. Similarly, saporin, an inhibitor of ribosomal assembly, can be coupled to antibodies via a disulfide bond between chemically inserted sulfhydryl groups. See Polito et al., Leukemia 18:1215-1222 (2004).

Radioisotopes can also be linked to antibodies, such as, for example, yttrium⁹⁰ (90Y), indium¹¹¹ (111In), ¹³¹I, ⁹⁹mTc, radiosilver-111, radiosilver-199, and Bismuth²¹³. Linkage of radioisotopes to antibodies may be performed with conventional bifunction chelates. For radiosilver-11 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1:S91-S95 (2001).

Other therapeutic agents may also be linked to antibodies. Therapeutic agents are usually cytotoxic or cytostatic. For example, antibodies can be conjugated with toxic chemotherapeutic drugs such as maytansine, geldanamycin, tubulin inhibitors, such as auristatins, or minor groove binding agents, such as calicheamicin. Other representative therapeutic agents include agents known to be useful for treatment, management, or amelioration of an autoimmune disease, a neuroinflammatory disease, or a cancer, or symptoms of an autoimmune disease, a neuroinflammatory disease, or a cancer. Examples of such therapeutic agents are disclosed elsewhere herein.

Antibodies can also be coupled with other proteins. For example, antibodies can be coupled with Fynomers. Fynomers are small binding proteins (e.g., 7 kDa) derived from the human Fyn SH3 domain. They can be stable and soluble, and they can lack cysteine residues and disulfide bonds. Fynomers can be engineered to bind to target molecules with the same affinity and specificity as antibodies. They are suitable for creating multi-specific fusion proteins based on antibodies. For example, Fynomers can be fused to N-terminal and/or C-terminal ends of antibodies to create bi- and tri-specific FynomAbs with different architectures. Fynomers can be selected using Fynomer libraries through screening technologies using FACS, Biacore, and cell-based assays that allow efficient selection of Fynomers with optimal properties. Examples of Fynomers are disclosed in Grabulovski et al., J. Biol. Chem. 282:3196-3204 (2007); Bertschinger et al., Protein Eng. Des. Sel. 20:57-68 (2007); Schlatter et al., MAbs. 4:497-508 (2011); Banner et al., Acta. Crystallogr. D. Biol. Crystallogr. 69(Pt6):1124-1137 (2013); and Brack et al., Mol. Cancer Ther. 13:2030-2039 (2014).

The antibodies disclosed herein can also be coupled or conjugated to one or more other antibodies (e.g., to form antibody heteroconjugates). Such other antibodies can bind to different epitopes within MCAM or can bind to a different target antigen.

Antibodies can also be coupled with a detectable label. Such antibodies can be used, for example, for diagnosing of an autoimmune disease, a neuroinflammatory disease, or a cancer, for monitoring progression of an autoimmune disease, a neuroinflammatory disease, or a cancer, and/or for assessing efficacy of treatment. Such antibodies can be useful for performing such determinations in subjects having or being susceptible to an autoimmune disease, a neuroinflammatory disease, or a cancer, or in appropriate biological samples obtained from such subjects. Representative detectable labels that may be coupled or linked to an antibody include various enzymes, such as horseradish peroxidase, alkaline phosphatase, beta-galactosidase, or acetylcholinesterase; prosthetic groups, such streptavidin/biotin and avidin/biotin; fluorescent materials, such as umbelliferone, fluorescein, fluorescein isothiocyanate, rhodamine, dichlorotriazinylamine fluorescein, dansyl chloride or phycoerythrin; luminescent materials, such as luminol; bioluminescent materials, such as luciferase, luciferin, and aequorin; radioactive materials, such as radiosilver-111, radiosilver-199, Bismuth²¹³, iodine (¹³¹I, ¹²⁵I, ¹²³I, ¹²¹I), carbon (¹⁴C), sulfur (⁵S), tritium (³H), indium (¹¹⁵In, ¹¹³In, ¹¹²In, ¹¹¹In,) technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies; nonradioactive paramagnetic metal ions; and molecules that are radiolabelled or conjugated to specific radioisotopes.

Therapeutic agents, other proteins, other antibodies, and/or detectable labels may be coupled or conjugated, directly or indirectly through an intermediate (e.g., a linker), to a murine, chimeric, veneered, or humanized antibody using techniques known in the art. See e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985); and Thorpe et al., Immunol. Rev., 62:119-58 (1982). Suitable linkers include, for example, cleavable and non-cleavable linkers. Different linkers that release the drugs under acidic or reducing conditions or on exposure to specific proteases can be employed. Likewise, different linkers that release the coupled therapeutic agents, proteins, antibodies, and/or detectable labels under acidic or reducing conditions, on exposure to specific proteases, or under other defined conditions can be employed.

### IV. Methods of Treatment

The antibodies or other antagonists disclosed herein can be used for treating or effecting prophylaxis of subjects having (e.g., meeting art-recognized criteria, such as those of the DSM-IV-TR or DSM-V) or at elevated risk relative to the general population of a disease disclosed herein. Elevated risk can be assessed from presence of one or more genetic or biochemical markers associated with the disease, or one or more symptoms consistent with the disease but insufficient to allow a definite diagnosis. Some specific exemplary diseases treatable by the present methods include giant cell arteritis, polymyalgia rheumatica (PMR) and Takayasu's arteritis. These diseases are characterized by infiltration of TH17 and TH1 cells into blood vessel walls. Cells expressing MCAM have been found to localize around blood vessels. Although practice of the methods is not dependent on understanding of mechanism, it is believed that in some methods antibodies or other antagonists function at least in part by inhibiting the interaction of MCAM expressed on T cells (e.g., TH17 cells) and laminin α4 chain, e.g., an α4 chain of laminin 411 expressed on the surface of an endothelial cell forming a blood vessel wall. Antibody-drug conjugates can have additional mechanisms of action including the cytotoxic or cytostatic effect of the linked agent, typically after uptake within the targeted cell. Antibody-drug conjugates may also induce macrophage toxicity.

Giant-cell arteritis (GCA or temporal arteritis or cranial arteritis or Horton disease) is an inflammatory disease of blood vessels most commonly involving large and medium arteries of the head, predominantly the branches of the external carotid artery. The most serious complication is occlusion of the ophthalmic artery, which is a branch of the internal carotid. It can create a medical emergency which can cause irreversible ischemia and blindness if not treated promptly. GCA is conventionally treated with glucocorticoids, which reduce current symptoms of inflammation and prevent occlusion but has no effect on vessel wall pathology, resulting in subjects relapsing after recovery.

GGA onset occurs at age 50 onward with a mean age of diagnosis of 72. About 190,000 subjects present with the disease. This disease frequently manifests as an analgesic resistant headache (60-90%) and visual loss (12-40%). Symptoms show a dramatic initial response to glucocorticoids.

GCA is diagnosed from clinical presentation, patient characteristics, blood test markers of inflammation and most characteristically, presence of giant multinucleate cells in vessel wall biopsies most usually proximate to the internal elastic membrane.

GCA is caused by infiltration of TH17 and TH1 cells into a blood vessel wall. Histo-pathological lesions are observed in all layers of the artery leading to segmental and focal panarteritis with a polymorphic cell infiltrate that includes T cells, macrophages and multinucleated giant cells, a fragmented internal elastic lamina and intimal hyperplasia. TH17 cells are the precursors of giant cells (Samson Clin Exp Rheumatol. 2013 January-February; 31(1 Suppl 75):565-73. Epub 2013 Apr. 19.). Therefore antibodies of the disclosure can inhibit GCA by inhibiting migration of TH17 or TH1 cells into blood vessel walls or conversion of TH17 cells to giant cells, among other mechanism. An animal model for GCA is available for testing antibodies. The animal model is formed by transferring human temporal arterial specimens subcutaneously into SCID mice (Deng et al., Circ. Res. 104, 488-495 (2009)). A cellular model is also available for analyzing the effect of potential drugs on migration and proliferation of smooth muscle cells in the vasculature. This models uses smooth muscle cells on a Matrigel™ matrix.

Polymyalgia rheumatica abbreviated as PMR, is a related condition to GCA, in which subjects have pain or stiffness, usually in the neck, shoulders, upper arms and hips, but which may occur all over the body. The pain can be very sudden, or can occur gradually over a period. It may be caused by an inflammatory condition of blood vessels such as temporal arteritis. Elevated ESR and C-reactive protein are characteristic of PMR as is a rapid response to low dose corticosteroids, which are the conventional treatment for PMR.

PMR can occur concurrently or separately than GCA. Approximately 15% of patients with PMR develop giant cell arteritis (GCA), and 40-50% of patients with GCA have associated PMR. Most inflammation is at the level of the synovium and bursae, with MRI studies revealing periarticular inflammation as well as bursitis in the bursae associated with both the shoulder and hip girdles. Systemic macrophage and T-cell activation are characteristic of both GCA and PMR. Patients often have an elevated IL-6 level which is likely responsible for the systemic inflammatory response in both GCA and PMR. A decrease in the level of circulating IL-6 correlates with remission of clinical symptoms. As with GCA, age of onset is 50 year or later with a mean of 72 years.

Takayasu's arteritis is a rare type of vasculitis related to GCA. It is a form of large vessel granulomatous vasculitis with massive intimal fibrosis and vascular narrowing, affecting often young or middle-aged women of Asian descent. It mainly affects the aorta (the main blood vessel leaving the heart) and its branches, as well as the pulmonary arteries. Females are about 8-9 times more likely to be affected than males. Those with the disease often notice symptoms between 15 and 30 years of age Takayasu's arteritis can also lead to arm or chest pain and high blood pressure and eventually to heart failure or stroke. The goal of treatment is to relieve inflammation in the arteries and prevent potential complications. Signs and symptoms of Takayasu's arteritis include: arm or leg weakness or pain with use (claudication), Lightheadedness or dizziness, fainting, headaches, memory problems, trouble thinking, shortness of breath, visual problems, high blood pressure, difference in blood pressure between arms, a difficult-to-find or absent pulse in the wrists; too few red blood cells (anemia), chest pain, abdominal pain. The condition is characterized by segmental and patchy granulomatous inflammation of the aorta and its major derivative branches. This inflammation leads to arterial stenosis, thrombosis, and aneurysms. There is also irregular fibrosis of the blood vessels due to chronic vasculitis, leading to sometimes massive intimal fibrosis (fibrosis of the inner section of the blood vessels). Prominent narrowing due to inflammation, granuloma, and fibrosis is often seen in arterial studies such as magnetic resonance angiography (MRA), computed tomography angiography (CTA), or arterial angiography (DSA). Treatments include corticosteroids, methotrexate, azathioprine, adalimumab, etenercept, infliximab and tocilizumab.

Antibodies or other antagonists are administered in an effective regime meaning a dosage, route of administration and frequency of administration that delays the onset, reduces the severity, inhibits further deterioration, and/or ameliorates at least one sign or symptom of a disease being treated (e.g., giant cell arteritis, polymyalgia rheumatica (PMR) or Takayasu's arteritis). If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an antibody are 0.1-20, or 0.5-5 mg/kg body weight (e.g., 0.5, 1, 2, 3, 4 or 5 mg/kg) or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. For some antibodies and under some circumstances, administration into the systemic circulation by intravenous or subcutaneous administration is preferred. Intravenous administration can be, for example, by infusion over a period such as 30-90 min.

The frequency of administration depends on the half-life of the antibody in the circulation, the condition of the patient and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

Treatment with antibodies or other antagonists disclosed herein can be combined with other treatments effective against the disorder being treated. Combination treatments can be formulated for administered separately.

### V. Formulations

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

The disclosure provides formulations comprise an antibody or other antagonist described herein, a buffer, one or more sugars and/or polyols and a surfactant, and have a pH within the range from about 5.5 to about 7. The formulations can be prepared for storage in liquid form or in lyophilized form. When stored in lyophilized form, the formulations can be reconstituted with a liquid (e.g., sterile water) to the concentrations and properties described herein. When a lyophilized composition is said to be reconstitutable by adding water to generate a formulation of specified component concentrations and pH, it is meant that the lyophilized formulation can be so reconstituted simply by addition of water (i.e., without supplying additional amounts of components or adding acid or base to change the pH). The concentrations and properties of a prelyophilized liquid formulation can also be in accordance with those described below if the lyophilized formulation is reconstituted to the same volume as the formulation prelyophilization. If the volume is different, then concentrations of formulations should be adjusted proportionally. For example, if the reconstituted volume is half the prelyophilization volume, then the concentrations of components in the prelyophilization formulation should be half the concentrations in the reconstituted formulation.

Optionally, the antibody is resuspended in a formulation as described below, temporarily frozen for storage prelyophilization, lyophilized, and reconstituted with water to the same concentrations as prelyophilization. Such a formulation should preferably stabilize the antibody throughout freezing, lyophilization, storage, and reconstitution as well as being suitable for parenteral administration. In an exemplary work flow, a purified antibody is resuspended at about 40 mg/mL in a formulation and stored frozen at −40° C. in bags. Bags are thawed at room temperature for 3 hours and the contents are pooled. The formulation is sterile filtered through a 0.2 micron sterile filer. Vials are filled with 5.4 mL of the formulation and lyophilized. Lyophilized vials are stored at 2-8° C. Lyophilized vials are reconstituted by adding sterile water (e.g., approximately 5.0 to 5.4 mL sterile water, depending on the formulation). Five mL of the reconstituted product is then added into the port of an IV bag containing 20-100 mL of normal saline, lactated Ringers solution, or 5% dextrose solution or the like for intravenous infusion into a patient.

Some formulations include a bulking agent, which may or may not be the same as the sugar/polyol component. Typically, the formulations are sterile, for example, as accomplished by sterile filtration using a 0.2 μm or a 0.22 μm filter. The formulations are also generally stable by low to undetectable levels of fragmentation and/or aggregation as further defined below on freezing and thawing. Still other formulations are stable following reconstitution of a lyophilized cake for at least three months at about 40° C. In some formulations, less than about 5% of the antibody is present as an aggregate in the formulation.

In some formulations, the antibody is present at a concentration within the range from about 5 mg/mL to about 100 mg/mL. In some formulations, the antibody is present at a concentration within the range from about 5 mg/mL to about 50 mg/mL. In some formulations, the antibody is present at a concentration within the range from about 25 mg/mL to about 50 mg/mL. For example, the antibody may be present at a concentration of about 35-45 mg/mL or about 40 mg/mL. The antibody may be present in a sterile liquid dosage form of about 50 mg/vial to about 500 mg/vial, or greater. The antibody may be present in a lyophilized dosage form of about 40 mg/vial to about 500 mg/vial. For example, the antibody may be present in a sterile liquid or lyophilized dosage form of about 250-350 mg/vial or about 200 mg/vial.

The formulation can comprise any of the antibodies described herein. In some formulations, the formulated antibody is an antibody comprising: (i) a mature heavy chain variable region comprising the three Kabat CDRs of SEQ ID NO:161 except that position 32 (Kabat numbering) can be N, S, or Q, and position 33 (Kabat numbering) can be G or A, wherein the mature heavy chain variable region is at least 90% identical to SEQ ID NO:161, and (ii) a mature light chain variable region comprising the three Kabat CDRs of SEQ ID NO:123, and being at least 90% identical to SEQ ID NO:123. In such formulations, position 1 (Kabat numbering) of the mature heavy chain variable region can be occupied by E. In some formulations, the mature heavy chain variable region has the amino acid sequence of SEQ ID NO:157, SEQ ID NO:158, SEQ ID NO:159, SEQ ID NO:160, or SEQ ID NO:161, and the mature light chain variable region has the amino acid sequence of SEQ ID NO:121, SEQ ID NO:122, or SEQ ID NO:123. For example, in some formulations, the mature heavy chain variable region has the amino acid sequence of SEQ ID NO:161 and the mature light chain variable region has the amino acid sequence of SEQ ID NO:123.

In other formulations, the formulated antibody is an isolated anti-MCAM antibody described herein. In such a formulation, the isolated anti-MCAM antibody binds to human MCAM (SEQ ID NO:11) at an epitope including amino acid residue 141.

Buffers are used in the disclosed formulations to achieve a suitable pH for the antibody, such as, for example, histidine, succinate, and citrate buffers. Some formulations have a pH within the range from about 5.5 to about 7, for example, a pH of 5.5, 5.6, 5.7, 5.8, 5.9, 6.0, 6.1, 6.2, 6.3, 6.4, 6.5, 6.6, 6.7, 6.8, 6.9, or 7.0. Some formulations have a pH of between about 5.5 to about 6.5. Some formulations have a pH of about 6.0 and other formulations have a pH of about 6.5. In some formulations, histidine buffer is present at a concentration within the range from about 10 mM to about 30 mM, for example, at a concentration of about 15-25 mM or about 20 mM.

Suitable sugars and/or polyols for the formulations include trehalose and sucrose, or a combination thereof. Sugars/polyols serve as bulking agents, lyoprotecting agent, and/or tonicity adjusting agents. For example, some formulations include trehalose present at a concentration within the range from about 200 mM to about 260 mM, or sucrose present at a concentration within the range from about 200 mM to about 260 mM. Some formulations include trehalose present at a concentration of about 220 mM. Other formulations include sucrose present at a concentration of about 220 mM. Some such formulations are characterized by an osmolality in the range of about 250-400, 300-400, or 300-350 mOsm/kg, such as, for example, 287 or 295 mOsm/kg.

Formulations can contain a surfactant to reduce antibody aggregation and absorption to surfaces. Suitable surfactants include polysorbate 20 present at a concentration within the range from about 0.005% to about 0.05% by weight. Polysorbate 20 protects against marked increases in aggregation or turbidity that would otherwise occur in formulations of antibodies. The polysorbate 20 may be present at a concentration within the range from about 0.01% to about 0.05%. For example, the concentration can be 0.005%, 0.01%, 0.015%, 0.02%, 0.025%, 0.03%, 0.035%, 0.04%, 0.045%, or 0.05%. Alternatively, in some formulations, polysorbate 20 is present at a concentration within the range of about from about 0.05 g/L, 0.1 g/L, 0.15 g/L, 0.2 g/L, 0.25 g/L, 0.3 g/L, 0.35 g/L, 0.4 g/L, 0.45 g/L, or 0.5 g/L. Some formulations include polysorbate 20 at a concentration of 0.2 g/L.

An exemplary formulation (liquid, prelyophilization or reconstituted after lyophilization) is characterized by a pH within the range from about 5.5 to about 7 and includes: (a) an antibody described herein, at a concentration within the range from about 10 mg/mL to about 50 mg/mL; (b) a histidine buffer present at a concentration within the range from about 10 mM to about 30 mM; (c) one or more sugars and polyols (“sugar/polyol”) selected from trehalose present at a concentration within the range from about 200 mM to about 260 mM, and sucrose present at a concentration within the range from about 200 mM to about 260 mM; and (d) polysorbate 20 present at a concentration within the range from about 0.005% to about 0.05% by weight. In one example, the formulation can include: (a) any antibody described herein; (b) a histidine buffer at a concentration of about 20 mM; (c) sucrose at a concentration of about 220 mM; (d) polysorbate 20 at a concentration of about 0.02%; and a pH of about 6.0. In another example, the formulation can include: (a) any antibody described herein; (b) a histidine buffer at a concentration of about 20 mM; (c) trehalose at a concentration of about 220 mM; (d) polysorbate 20 at a concentration of about 0.02%; and a pH of about 6.5.

Some lyophilized formulations include: (a) an antibody described herein; (b) histidine buffer; (c) trehalose or sucrose; and (d) polysorbate 20. The lyophilized formulation can include about 200 mg of the antibody. Some lyophilized formulations are capable of being reconstituted with sterile water. Some lyophilized formulations include 100-300 or 150-250 mg antibody, 10 to 20 or 14 to 16 mg of histidine, 300 to 450 or 350 to 400 mg sucrose, and 0.5 to 1.5 mg or 0.75 to 1.25 mg polysorbate 20. Other lyophilized formulations include 100 to 300 or 150 to 250 mg antibody, 10 to 20 or 14 to 16 mg of histidine, 360 to 500 or 400 to 450 mg trehalose dehydrate, and 0.5 to 1.5 mg or 0.75 to 1.25 mg polysorbate 20.

An exemplary lyophilized formulation includes 200 mg of an antibody, 15.5 mg of histidine, 376 mg sucrose, and 1 mg polysorbate 20. Another exemplary lyophilized formulation includes 200 mg of an antibody, 15.5 mg of histidine, 416 mg trehalose dihydrate, and 1 mg polysorbate 20. Some such formulations can be reconstituted to a volume of about 5 mL. Other lyophilized formulations include the same components in the same proportions as any disclosed in this paragraph but in different amounts (e.g., 400 mg antibody, 31 mg histidine, 752 mg sucrose, and 2 mg polysorbate 20).

Lyophilized formulations can be reconstituted to an antibody concentration of about 30-50 or 35-45 mg/mL, for example to about 40 mg/mL; (b) a histidine buffer present at a concentration of about 10-30 or 15-25 mM, for example about 20 mM; (c) sucrose or trehalose present at a concentration of about 160-330 or 200-260 mM, for example about 220 mM; (d) polysorbate 20 present at a concentration of about 0.1-0.3 or 0.15 to 0.25 g/L, for example about 0.2 g/L; and (e) a pH of about 5.5-6.5, for example about 6.0 (if sucrose is present) or 6.5 (if trehalose is present).

Liquid or reconstituted lyophilized formulations are preferably substantially isotonic, implying an osmolality of about 250-350 mOsm/kg water. Some formulations have an osmolality of 270-300 mOsm/kg. Some formulations have an osmolality of about 287 or about 295 mOsm/kg. Liquid or reconstituted lyophilized formulations can also be hypertonic >350 mOsm/kg water or hypotonic (<250 mOsm/kg water).

Any of the formulations described can be made without pharmaceutical excipients, carriers or the like, other than those described as being components herein. Such a formulation can be described as consisting of the recited components, or consisting essentially of the recited components if insignificant amounts of other components not affecting the properties of the formulation are present. Formulations are preferably made under good manufacturing practices (GMP) approved or approvable by the FDA for preparation of drugs for administration to humans.

The disclosure encompasses antibody formulations having stability at 38° C.-42° C. (e.g., as assessed by high performance size exclusion chromatography (HPSEC)) for at least about 30 days, for at least about 3 months, or longer. Such formulations may also have stability at 20° C.-24° C. for at least about 1 year, and/or stability at 2° C.-4° C. for at least about 3 years. Stability of lyophilized formulations is assessed for storage in the lyophilized state. A formulation is considered stable if, after incubation at one or more of these specified combinations of time and temperature, it meets the below definition for low to undetectable fragmentation and/or low to undetectable aggregation. More particularly, the disclosed formulations exhibit low to undetectable levels of antibody aggregation and/or fragmentation, or a low or undetectable increase in fragmentation and/or aggregation above an initial level (e.g., less than about 5% aggregation). A formulation having low to undetectable levels of fragmentation contains at least about 65%, 70%, 75%, 80%, 85%, 90%, 95%, 98%, or 99%, of the total protein, for example, in a single peak as determined by hydrophobic interaction chromatography, or in two peaks (one corresponding to each of the antibody heavy chains and antibody light chains) by reduced Capillary Gel Electrophoresis (rCGE), representing the non-degraded antibody, and containing no other single peaks having more than 5%, more than 4%, more than 3%, more than 2%, more than 1%, or more than 0.5% of the total protein each. A formulation having low to undetectable levels of aggregation contains no more than about 15%, no more than about 10%, no more that about 5%, no more than about 4%, no more than about 3%, no more than about 2%, no more than about 1%, or no more than about 0.5% aggregation by weight protein, as measured by high performance size exclusion chromatography (HPSEC). For example, in some formulations, less than about 5% of the antibody is present as an aggregate. Stable formulations also show little or no loss of biological activity(ies) having, for example, binding affinity measurable by ELISAs and/or additional functional assay, that is at least about 50%, 55%, 60%, 65%, 70%, 75%, 80%, 85%, 90%, 95%, 98%, or 99% of an initial measurable value.

### VI. Kits

The disclosure further provides kits (e.g., containers) comprising the MCAM antibodies or other antagonists disclosed herein and related materials, such as instructions for use (e.g., package insert). The instructions for use may contain, for example, instructions for administration of the MCAM antagonists and optionally one or more additional agents. The containers of MCAM antagonist(s) may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products.

Kits can also include a second container comprising a pharmaceutically-acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Materials and Methods

### Antibody Generation/Characterization

For the generation of antibodies capable of binding to murine MCAM, MCAM-Fc was generated by fusing the extracellular domain of murine MCAM to human IgG and produced in CHO cells using standard techniques. Lou/M rats were immunized with 100 μg of MCAM-Fc protein in CFA (1:1 volume). Rats were boosted two times at two week intervals with MCAM-Fc protein in incomplete Freund's adjuvant (IFA) (1:1 volume). Hybridomas were generated from immunized rats using standard protocols and clones were selected by Clonepix. CHO cells were transfected with the full length murine MCAM gene and selected for stable expression using neomycin and standard techniques. Parental CHO cells (MCAM negative) were fluorescently labeled with carboxyfluorescein succinimidyl ester (CFSE) using standard techniques and mixed at a 1:1 ratio with unlabeled MCAM transfected CHO cells. Hybridoma supernatants were incubated with this mixture of cells for 30 minutes and binding of potential MCAM specific antibodies was detected with a fluorescently labeled anti-rat secondary antibody (Jackson Immuno) by flow cytometry.

Supernatants from hybridomas that screened positive for MCAM specific antibodies were pre-incubated with fluorescently labeled mouse MCAM-Fc protein (5 μg/mL) for 30 minutes before addition to the laminin α4 expressing cell line WM2664 and neutralization of binding of the MCAM-Fc protein to the cell line was determined by flow cytometry.

For the generation of rat antibodies capable of binding to human MCAM, hMCAM-Fc was generated by fusing the extracellular domain of human MCAM to human IgG and produced in CHO cells using standard techniques. Lou/M rats were immunized with 250 μg of hMCAM-Fc protein in CFA (1:1 volume). Rats were boosted two times at two week intervals with hMCAM-Fc protein in incomplete Freund's adjuvant (IFA) (1:1 volume). Hybridomas were generated from immunized rats using standard protocols and clones were selected by Clonepix. CHO cells were transfected with the full length human MCAM gene and selected for stable expression using neomycin and standard techniques. Parental CHO cells (MCAM negative) were fluorescently labeled with carboxyfluorescein succinimidyl ester (CFSE) using standard techniques and mixed at a 1:1 ratio with unlabeled human MCAM transfected CHO cells. Hybridoma supernatants were incubated with this mixture of cells for 30 minutes and binding of potential human MCAM specific antibodies was detected with a fluorescently labeled anti-rat secondary antibody (Jackson Immuno) by flow cytometry.

For the generation of mouse antibodies capable of binding to human MCAM, hMCAM-Fc was generated by fusing the extracellular domain of human MCAM to human IgG and produced in CHO cells using standard techniques. Balb/c mice were immunized with 50 μg of hMCAM-Fc protein in CFA (1:1 volume). Mice were boosted two times at two week intervals with hMCAM-Fc protein in incomplete Freund's adjuvant (IFA) (1:1 volume). Hybridomas were generated from immunized mice using standard protocols and clones were selected by Clonepix. CHO cells were transfected with the full length human MCAM gene and selected for stable expression using neomycin and standard techniques. Parental CHO cells (MCAM negative) were fluorescently labeled with carboxyfluorescein succinimidyl ester (CFSE) using standard techniques and mixed at a 1:1 ratio with unlabeled human MCAM transfected CHO cells. Hybridoma supernatants were incubated with this mixture of cells for 30 minutes and binding of potential human MCAM specific antibodies was detected with a fluorescently labeled anti-mouse secondary antibody (Jackson Immuno) by flow cytometry.

Supernatants from hybridomas that screened positive for human MCAM specific antibodies were pre-incubated with fluorescently labeled hMCAM-Fc protein (5 μg/mL) for 30 minutes before addition to the laminin α4 expressing cell line WM2664 and neutralization of binding of the hMCAM-Fc protein to the cell line was determined by flow cytometry.

Nucleic Acid and Protein Manipulation

For determination of CDRs, total RNA was isolated from hybridoma cells using RNAquous-4PCR kit (Ambion), and was used for cDNA synthesis. First and second strand cDNA was synthesized using methods modified from Marathon cDNA amplification (Clontech) with the cDNA adaptor ligated to the 5′-end of the obtained dscDNA. The reverse specific primer was designed based on the specific antibody isotype constant region sequence for both heavy and light chains, and was used along with the adaptor primer in the PCR amplification of both VL and VH fragments using Pfu Ultra DNA polymerase (Stratagene). The amplified PCR product was cloned into pCR-Blunt-TOPO (Invitrogen), and the nucleotide sequence was determined. The sequences of the identified clones were compared for percent identity within the VL and VH sequences.

For determination of IL-17 concentrations in the supernatant, ELISA was performed using a commercial kit (R&D Systems).

### Example 1. Generation of Anti-MCAM Monoclonal Antibodies

Mouse and rat monoclonal antibodies directed against human MCAM protein were generated as described in Materials and Methods above. The specific binding between the monoclonal antibody and human MCAM was confirmed by assessing the monoclonal antibody's ability to bind to cells transfected with human MCAM. For this, untransfected cells were labeled with carboxyfluorescein succinimidyl ester (CFSE) and mixed with unlabeled human MCAM transfected cells. Untransfected cells could, therefore, be differentiated.

Using these techniques, 823 independent mouse fusions clones were isolated and shown to express an antibody capable of binding to human MCAM. Additionally, 152 independent rat fusions clones were isolated and shown to express an antibody capable of binding to human MCAM.

Next, the anti-human MCAM monoclonal antibodies were used to test their ability to block the binding of human MCAM to its ligand. Human MCAM-Fc protein (5 μg/mL) was pre-incubated with isotype control antibody, or 10 μg/mL of the test monoclonal antibody for 30 minutes in PBS. The mixture was added to healthy spinal cord tissue sections and subsequently characterized by fluorescence microscopy as described in Materials and Methods above. Furthermore, parental CHO cells (CHOK1) or CHO cells transfected with a human MCAM gene were preincubated with CHO culture media (DMEM), recombinant laminin 411 (10 μg/ml), or recombinant laminin 511 (i.e., laminin 10 (α5β1γ1)) (10 μg/ml) at 37° C. for 45 minutes. Cells were washed, and specific binding of laminin 411, but not laminin 511, to MCAM was detected with a pan-laminin antibody by flow cytometry. Pre-incubation of human MCAM transfected CHO cells with the anti-MCAM antibody (at 20 μg/ml), prior to laminin incubation, abolished the binding of human MCAM to laminin 411.

Using this technique, it was shown that 87 of the 823 independent mouse fusion clones and 26 of the 152 independent rat fusion clones described above expressed an antibody that was capable of blocking the interaction between human MCAM protein and its ligand, α-4 chain of laminin.

### Example 2. Further Characterization of Anti-MCAM Monoclonal Antibodies

The 87 independent mouse fusion clones and 26 independent rat fusion clones described in Example 1 above as being capable of (i) binding to human MCAM, and (ii) blocking the interaction between human MCAM and the α-4 chain of laminin were further characterized as follows. First, IC50 quantitation for the ability of the monoclonal antibody to block the binding of human MCAM to the α-4 chain of laminin was determined as follows. CHO cells expressing human MCAM were incubated with an anti-human MCAM antibody (at various concentrations) for 30 minutes at 4 degrees Celsius. Unbound antibody was then washed away, and the cells were incubated with recombinant human laminin 411 at 20 ug/ml for 45 minutes at 37 degrees Celsius. Unbound laminin was then washed away, and the laminin bound to the surface of the cells was detected with fluorescently labeled anti-laminin antibodies. After washing, the amount of laminin bound to the surface was detected by flow cytometry, and IC50s were calculated based on the mean fluorescent intensity.

Using the above described assay, six independent anti-human MCAM monoclonal antibody clones were identified as binding to human MCAM and having the greatest ability to block the interaction between human MCAM expressed on the surface of cells and its binding ligand, human laminin 411. These six anti-MCAM monoclonal antibody clones are herein referred to as (i) the mouse anti-human MCAM monoclonal clones 1174.1.3, 1414.1.2, 1415.1.1, and 1749.1.3, and (ii) the rat anti-human MCAM monoclonal antibody clones 2120.4.19 and 2107.4.10. Amino acid and nucleic acid sequences of the heavy and light chains of these antibodies, and their hypervariable regions, are provided in SEQ ID NOs:29-92. More specifically, in the above assay, IC50s for the monoclonal antibody clones 1174.1.3, 1414.1.2, 1415.1.1, 1749.1.3, 2120.4.19, and 2107.4.10 were determined to be 0.469 ug/ml, 0.431 ug/ml, 0.307 ug/ml, 0.545 ug/ml, 0.888 ug/ml, and 0.290 ug/ml, respectively. Moreover, experiments performed to determine the specific binding affinity of each monoclonal antibody demonstrated that each was capable of binding to human MCAM protein with high affinity (data not shown). As such, each of these specific monoclonal antibodies was very capable of binding to human MCAM and inhibiting the interaction of cell-expressed human MCAM with its α-4 laminin binding ligand. In contrast, two control antibodies, a non-specific human IgG1 antibody and a previously described, fully human anti-MCAM antibody referred to as ABX-MA1 (e.g., see Mills et al., Cancer Res. 62:5106 (2002), and U.S. Pat. Nos. 6,924,360, 7,067,131, and 7,090,844) were both incapable of blocking the binding interaction between human MCAM and its laminin 411 counterpart. As such, the six specific monoclonal antibodies identified above possess the novel ability to both (i) bind with high affinity to human MCAM on the surface of living cells, and (ii) block the interaction of cell expressed human MCAM with a laminin protein comprising an α-4 laminin polypeptide chain.

### Example 3. Domain Binding Analysis for Anti-MCAM Monoclonal Antibodies

ForteBio analysis was employed to determine the location of the antigen epitope on the human MCAM protein that is recognized and bound by monoclonal antibody clones 1174.1.3, 1414.1.2, 1415.1.1, 1749.1.3, 2120.4.19, and 2107.4.10. The following protocol was used: ForteBio anti-human IgG Fc biosensors were used to immobilize various MCAMhFc domains including full length MCAMhFc protein on to biosensor surface. These sensors were dipped into the MCAM specific 1174.1.3, 1414.1.2, 1415.1.1, 1749.1.3, 2120.4.19, or 2107.4.10 antibody for detection of binding to these domains or full length protein. After loading these samples into a black 96 well plate, the Octet Red was programmed as follows: 60 seconds for baseline #1; 180 seconds for loading various domains; 60 seconds for baseline #2; 180 seconds for association of antibody to domain; and 240 seconds for dissociation of antibody from domain.

Reagents and Supplies Used:
1. MCAMhFc final concentration @ 5 ug/ml
2. antibody clones 1174.1.3, 1414.1.2, 1415.1.1, 1749.1.3, 2120.4.19, and 2107.4.10 clones @5 ug/ml
3. ForteBio anti-human IgG Fc Capture (AHC) biosensors for kinetics experiments, cat#18-5060
4. Block 96 well plate from Greiner Bio-one, cat#655209
5. ForteBio Octet Red machine
6. Fresh tissue culture medium, DMEM with 20% FCS, was used as buffer for dilution

The results from these analyses are as follows.

Monoclonal antibody clones 1174.1.3, 1414.1.2, 1415.1.1, and 1749.1.3 were all shown to bind to an antigenic epitope found on domain 3 of the human MCAM protein, defined specifically by amino acids 244-321 (SEQ ID NO:24) of the human MCAM protein. These monoclonal antibodies were not capable of binding to human MCAM domain 1 (namely amino acids 19-129, SEQ ID NO:22), domain 2 (namely amino acids 139-242, SEQ ID NO:23), or the combination of domains 1 and 2 (namely, amino acids 19-242). Hence, monoclonal antibody clones 1174.1.3, 1414.1.2, 1415.1.1, and 1749.1.3 define a novel antigenic epitope located within domain 3 of the human MCAM protein.

Monoclonal antibody clones 2120.4.19, and 2107.4.10 were each shown to bind to an antigenic epitope defined by the combination of human MCAM domain 1 (namely amino acids 19-129, SEQ ID NO:22), and domain 2 (namely amino acids 139-242, SEQ ID NO:23). Neither of these two monoclonal antibodies bound to human MCAM domain 1 by itself. Hence, monoclonal antibody clones 2120.4.19 and 2107.4.10 define a novel antigenic epitope determined by the presence of both human MCAM protein domains 1 and 2.

In contrast to the above, the previously described fully human anti-MCAM antibody ABX-MA1 binds to a different antigenic epitope than those described above, namely an antigenic epitope that is fully defined and encompassed within human MCAM domain 1 only.

Given these results, since each of monoclonal antibody clones 1174.1.3, 1414.1.2, 1415.1.1, 1749.1.3, 2120.4.19, and 2107.4.10 are capable of both (i) binding to human MCAM, and (ii) blocking the interaction between human MCAM and an α-4 laminin containing protein, whereas the ABX-MA1 antibody is capable of only binding to human MCAM, but not blocking the interaction between human MCAM and an α-4 laminin containing protein, these results demonstrate that human MCAM domain 2, human MCAM domain 3, and the combination thereof play a role in the binding interaction with α-4 laminin chain. Given this, it is clear that antibodies which bind to human MCAM domain 2, human MCAM domain 3, and/or the combination thereof would find use as agents capable of blocking the interaction between human MCAM and α-4 laminin and, thereby, find use for inhibiting the various consequences described herein resulting from that interaction. In contrast, antibodies that bind to an antigenic epitope defined solely by human MCAM domain 1 (such as the ABX-MA1 antibody described herein) are not useful for blocking the MCAM/α-4 laminin interaction and its various downstream biological consequences.

### Example 4. Shotgun Mutagenesis Epitope Mapping

Various amino acid residue of interest for anti-MCAM antibody binding were identified using shotgun mutagenesis and high-throughput cellular expression technology that enables the expression and analysis of large libraries of mutated target proteins within eukaryotic cells. Every residue in the human MCAM protein was individually mutated to an alanine, or other specified residue, to assay changes in function. Proteins were expressed within standard mammalian cell lines.

Table 1 shows a summary of the reagents and methods used to generate the shotgun mutagenesis library.

TABLE 1

| | |
|---|---|
| Parental plasmid | hsMCAM-V5/HIS6 (Accession # NP 006491) |
| Final library size | 528 mutant clones plus 17 additional site-directed |
| | mutants |
| Mutation Strategy | Alanine Scanning Mutagenesis |
| Cell type | BHK-S |
| Epitope Tag | C-terminal V5/HIS6 |

Full-length human MCAM was successfully codon-optimized, synthesized, and subcloned into a mammalian high-expression vector. This parental construct was then sequence-verified and validated for mammalian cell expression by immunodetection methods.

Detection of 2120.4.19 antibody and mouse sera binding to MCAM by immunofluorescence was successfully optimized for the high-throughput shotgun mutagenesis format. Serial dilutions of each primary antibody were tested with a single dilution of secondary antibody in a 384-well format. Antibodies were tested for detection of 293T and BHK cells expressing human MCAM. Optimal assay conditions were selected for screening the complete mutation library.

The MCAM mutation library was created and sequence verified, consisting of 545 clones (528/536 alanine mutants and 17/17 site-directed mutants), each bearing either a single residue substitution to alanine (alanine residues are substituted to serine) or a specified residue. Residues 35, 66, 161, 261, 342, 380, 414, and 435 are not represented in the library. The mutation library was screened in triplicate by immunodetection for binding to mouse sera. This validates cell surface expression for each mutant clone.

Multiple rounds of optimization were performed to determine conditions that are suitable for mapping. The following variables were evaluated: multiple laminin concentrations and anti-laminin secondary antibody concentrations, various blocking buffers to reduce nonspecific binding, multiple cell types, and multiple washing steps.

The mutation library was screened in triplicate by immunodetection for binding to the 2120.4.19 antibody. Reactivity was quantified for each mutant to identify point mutants that exhibit loss of binding.

Monoclonal antibody and sera reactivity were quantified for each mutant clone to identify point mutants that exhibit loss of binding without impacting surface expression. The critical residues for each antibody were identified by comparison of the monoclonal antibody binding profile to the sera binding profile of each mutant clone.

BHK cells were transfected with either wild-type (WT) MCAM or vector alone in a 384-well format, followed by immunodetection. Serial dilutions of each antibody (beginning with 4 μg/ml) were tested for immunoreactivity against WT or vector alone (Table 2). Each point represents the average of four replicates.

TABLE 2

| Primary | | | |
|---|---|---|---|
| Ab | MAb | | Ms Sera |
| conc | 2120.4.19 | Ms Sera | Conc |

| (ug/mL) | S/B | Z′ | S/B | Z′ | (ug/mL) |
|---|---|---|---|---|---|
| 4.00 | 11.48 | 0.54 | 6.4 | 0.19 | 1:100 |
| 2.00 | 22.92 | 0.56 | 7.6 | 0.53 | 1:200 |
| 1.00 | 32.46 | 0.63 | 8.3 | 0.74 | 1:400 |
| 0.50 | 36.87 | 0.43 | 7.91 | 0.55 | 1:800 |
| 0.25 | 36.99 | 0.41 | 11. | 0.50 | 1:1600 |
| 0.13 | 25.72 | 0.66 | 16. | 0.50 | 1:3200 |
| 0.06 | 15.79 | 0.67 | 10. | 0.54 | 1:6400 |
| 0.03 | 8.47 | 0.62 | 10. | 0.39 | 1:12800 |
| 0.02 | 4.95 | 0.65 | 7.2 | −0.19 | 1:25600 |
| 0.00 | 0.96 | −4.87 | 1.77 | −5.95 | 0.00 |

Optimal screening conditions for the immunodetection and epitope mapping of 2120.4.19 and Ms Sera were determined. Using these conditions, each antibody demonstrated a robust signal, high signal-to-background values, and low variability between replicates. These data indicate that these conditions are suitable for successful high-throughput epitope mapping. Final screening concentrations of 0.25 μg/mL for 2120.4.19 and a 1:800 dilution of the Ms Sera were used. Secondary antibodies from Jackson ImmunoResearch were used at 1:400 for 2120.4.19 and sera detection. Table 3 shows the experimental parameters optimized for high-throughput immunodetection.

TABLE 3

| Experimental | | |
|---|---|---|
| Parameter | MAb 2120.4.19 | Ms Sera |
| Cells Fixatie | BHK-S 4% PFA | BHK-S 4% PFA |
| Blocking Buffer | 10% Goat Serum | 10% Goat Serum |
| Primary Ab | 2120.4 | Sera |
| Ab name Target | MCAM | MCAM |
| Optimal Conc. | 0.25 ug/ml | 1:800 dilution |
| Incubation (RT) | 60 min | 60 min |
| Secondary Ab | | |
| Target | Rat IgG | Mouse IgG |
| Optimal Conc. | 1:400 (3.75 ug/mL) | 1:400 (3.75 ug/ml) |
| Incubation | 30 min | 30 min |
| Manufacturer | Jackson/ImmunoResearch | Jackson/ |
| | | ImmunoResearch |
| Cat # | 112-545-003 | 115-545-003 |
| Antibody ID | Alexa Fluor ® 488- | Alexa Fluor ® 488- |
| | AffiniPure | AffiniPure Goat Anti- |
| | Goat Anti-Rat IgG (H + L) | Mouse IgG (H + L) |
| Washes | PBS (CA²⁺, Mg²⁺ free) | PBS (CA²⁺, Mg²⁺ |
| | | free) |
| Signal:Background | 36:1 | 8:1 |

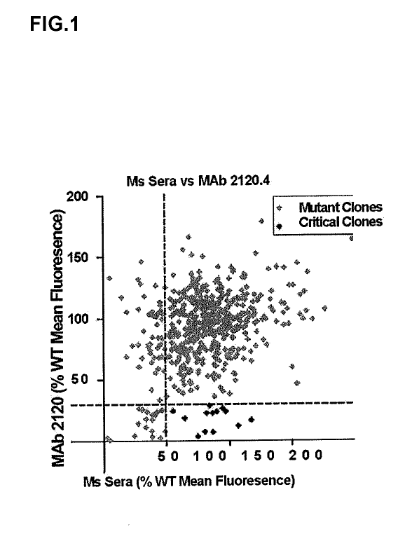

The mutation library was assayed for surface expression (mouse sera binding) and monoclonal antibody binding, in triplicate. Each raw data point was background-subtracted and normalized to the wild type MCAM reactivity values. The results are shown in FIG. 1. The mean monoclonal antibody binding value for 2120.4.19 is plotted as a function of its mean surface expression value (FIG. 1, gray diamonds). Thresholds of <30% monoclonal antibody reactivity and >50% mouse sera binding were applied to identify clones (FIG. 1, black diamonds) that were negative for monoclonal antibody binding but positive for surface expression.

Critical residues for 2120.4.19 were identified by evaluating the mean monoclonal antibody reactivity of each clone compared to its overall surface expression (average serum reactivity) (Table 4). Residues involved in antibody binding were identified as those that were negative for monoclonal antibody binding (<30% WT) but positive for surface expression (>50% WT). The mean reactivity (and standard deviation) are shown for each critical residue.

TABLE 4

| Residue ID | Mutations | 6MAb 2120.4 | | Ms Sera | |
|---|---|---|---|---|---|
| 39 | E39A | 22.6 | (16.2) | 86.8 | (9.2) |
| 62 | W62A | 7.1 | (4.7) | 86.8 | (12.6) |
| 133 | Y133A | 26.4 | (3.2) | 94.6 | (17.2) |
| 141 | I141A | 16.8 | (4) | 117.4 | (20.9) |
| 159 | A159S | 23.9 | (7.4) | 97 | (1.5) |
| 212 | L212A | 23.7 | (3.4) | 89.9 | (33.1) |
| 220 | Q220A | 22.5 | (10.9) | 81.9 | (36.1) |
| 221 | F221A | 3.7 | (0.6) | 75.1 | (65.2) |
| 223 | C223A | 7.2 | (2.5) | 80.7 | (25.6) |
| 227 | Y227A | 12.3 | (6.5) | 107.3 | (36.4) |
| 238 | S238A | 28.9 | (18.3) | 84.4 | (28.5) |
| 241 | V241A | 24.4 | (20.1) | 55.1 | (22.7) |
| 392 | L392A | 18.3 | (2.1) | 64.3 | (10.8) |

Potentially Critical Residues

| | | | | | |
|---|---|---|---|---|---|
| 145 | P145A | 26.3 | (6.7) | 28.3 | (4) |
| 167 | Y167A | 14.3 | (6.1) | 27.5 | (18.6) |
| 175 | Y175A | 7.7 | (8.1) | 49.1 | (5.9) |
| 206 | S206A | 17.9 | (2.7) | 33.5 | (13) |
| 207 | I207A | 23.6 | (6.2) | 42.8 | (26.8) |
| 216 | D216A | 8.2 | (2.7) | 38.4 | (19.5) |
| 225 | L225A | 23.8 | (17) | 34.8 | (1.9) |

The critical amino acids identified by shotgun mutagenesis mapping suggest binding sites for the and 2120.4.19 antibody. The data indicate that 2120.4.19 binds a conformationally complex epitope, while primarily binding the second Ig domain.

Critical residues appear largely dependent upon structural stabilization contributed by disulfide bonds of the second and/or third Ig domains. Binding for 2120.4.19 is supported by a cluster of critical residues that include one or both of the disulfide-bonded cysteines 161 and 223 of the second Ig domain.

### Example 5. Confirmatory MCAM Epitope Mapping for Antibody and Laminin Binding

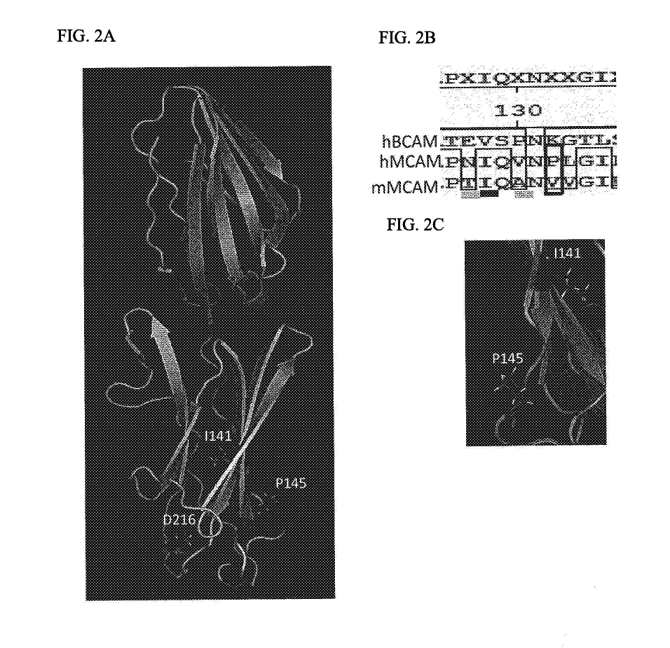

In order to identify binding sites of 2120.4.19 on human MCAM, a homology model of human MCAM Ig1 and Ig2 was built up on human BCAM Ig1 and Ig2 model by using Schrodinger Maestro (FIG. 2A). Twenty point mutants based on the structure information and shotgun mutagenesis information were designed and generated. These mutants were displayed on mammalian cells and FACS was used to test the binding of 2120.4.19 and laminin α-4 to the MCAM mutants. Three MCAM single mutants, I141A, D216A and Y318A, demonstrated a complete loss of laminin α-4 binding. I141A demonstrated complete loss of 2120.4.19 binding and P145V demonstrated significant loss of 2120.4.19 binding.

To further confirm the data, stable cell lines expressing I141A, P145V, D216A and Y318A respectively were generated. ForteBio assays were performed with the purified proteins as described above. The control ABX-MA1 antibody bound to wild type MCAM and the MCAM mutants. 2120.4.19 did not demonstrate significant binding to the MCAM I141A mutant. In addition, 2120.4.19 demonstrated greatly reduced binding to the MCAM P145V and D216A mutant respectively. Also, binding of 2120.4.19 to MCAM mutant P145V demonstrated a rapid K off.

### Example 6. Generation of Humanized Anti-MCAM 2120 Antibodies

Various humanized anti-MCAM antibodies were generated according to the following protocol. First, a three-dimensional molecular model of the variable regions was constructed using JN Biosciences' proprietary algorithm. Second, the framework amino acid residues important for the formation of the CDR structure or necessary for the binding to antigen were identified using the molecular model. In parallel, cDNA-derived human VH and VL amino acid sequences with high homology to the VH and VL amino acid sequences, respectively, were selected. Lastly, CDR sequences together with framework amino acid residues important for CDR structure or antigen binding were grafted from VH and VL into the corresponding selected human framework sequences.

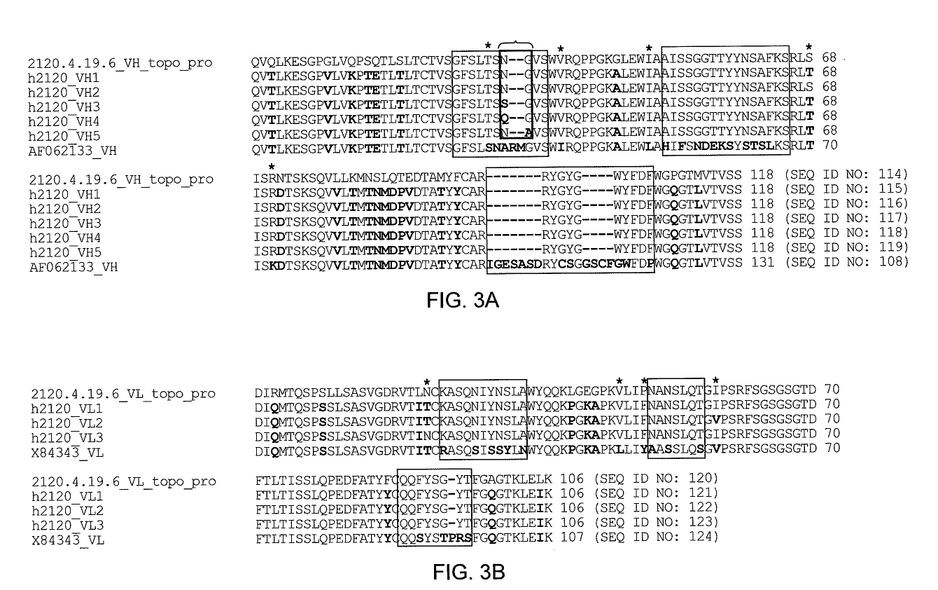

FIG. 3 depicts the alignment of various 2120 heavy and light chain sequences. Residue numbering is according to Kabat numbering. Different mutations to the framework (FR) amino acid residues involved in CDR formation and antigen binding were identified depending upon the version of antibody.

Exemplary mutations of the 2120 antibodies are depicted in FIG. 3A (boxed residues in CDR-H1 (S30T), between CDR-H1 and CDR-H2 (I37V and L48I), and between CDR-H2 and CDR-H3 (K71R) affect CDR contact; and S30T, I37V, L48I, and K71R mutations combined with an additional mutation after CDR-H2 (T68S) affect CDR contact); and FIG. 3B (boxed residues between CDR-L1 and CDR-L2 (L46V and Y49F) and between CDR-L2 and CDR-L3 (V58I) affect CDR contact; boxed residues between CDR-L1 and CDR-L2 (L46V and Y49F) affect CDR contact; and L46V, Y49F, and V58I mutations combined with an additional mutation before CDR-L1 (T22N) affect antibody/antigen interaction).

Several versions of each chain were designed (standard vs. aggressive or conservative). For those antibodies that contained N-deamidation motifs (NG), mutations to the asparagines or glycine were introduced into the standard version. The various humanized V regions were synthesized with a heterologous signal sequence and cloned into expression vectors containing human CK (VL) or human IgG1 (VH).

The heavy and light chain plasmids were co-transfected into 293F cells with the FreeStyle™ MAX transfection regent (Invitrogen) according to the manufacturer's protocol. The expressed antibody was purified with protein A PhyTip columns (Phynexus) and quantified via OD280.

The apparent affinities of the humanized antibodies were compared to the parental rodent or chimeric antibody in a competitive ELISA according to the following protocol.

ELISA plates were coated with recombinant hMCAM-His, and blocked with casein buffer to prevent non-specific binding. Biotinylated rodent or chimeric antibody was added at a subsaturating concentration, in the presence or absence of 3× increasing concentrations of unlabeled competitor (humanized antibody, rodent, or chimeric). After washing to remove unbound antibody, streptavidin HRP was added to allow detection of the biotinylated antibody. The ELISA was developed with TMB substrate and the OD450 was measured. The IC50 of the unlabeled competitor was determined using the GraphPad Prism5 software.

Table 5 summarizes the design of humanized sequences.

TABLE 5

| 2120 | Donor Framework | Mutations |
|---|---|---|
| VH1 | AF062133 IGHV2-26*01 | S30T*, I37V, L48I and K71R |
| VH2 | AF062133 IGHV2-26*01 | VH1 mutations + T68S |
| VH3 | AF062133 IGHV2-26*01 | VH1 mutations + N32S |
| VH4 | AF062133 IGHV2-26*01 | VH1 mutations + N32Q |
| VH5 | AF062133 IGHV2-26*01 | VH1 mutations + G33A |
| VL1 | X84343 IGKV1-39*01 | L46V, Y49F and V58I |
| VL2 | X84343 IGKV1-39*01 | L46V, Y49F |
| VL3 | X84343 IGKV1-39*01 | VL1 + T22N |

The heavy and light chain plasmids were co-transfected into 293F cells with the FreeStyle™ MAX transfection regent (Invitrogen) according to the manufacturer's protocol. The expressed antibody was purified with protein A PhyTip columns (Phynexus) and quantified via OD280.

The apparent affinities of the humanized antibodies were compared to the parental rodent or chimeric antibody in a competitive ELISA according to the following protocol: ELISA plates were coated with recombinant hMCAM-His, and blocked with casein buffer to prevent non-specific binding. Biotinylated rodent or chimeric antibody was added at a subsaturating concentration, in the presence or absence of 3× increasing concentrations of unlabeled competitor (humanized antibody, rodent, or chimeric). After washing to remove unbound antibody, streptavidin HRP was added to allow detection of the biotinylated antibody. The ELISA was developed with TMB substrate and the OD450 was measured. The IC50 of the unlabeled competitor was determined using the GraphPad Prism5 software.

The affinities were measured using the ForteBio Octet Red. Anti-human Fc sensors were used to capture the humanized antibodies, and several concentrations of hMCAMHis analyte were used to determine the affinity using a 1:1 fitting model.

The potencies of the antibodies were measured in the laminin/FACS assay according to the following protocol: recombinant laminin 411 (Biolaminate) was added to hMCAM expressing CHO cells in the presence or absence of varying concentrations of the humanized, rodent, or chimeric antibodies. Following incubation for 30-45 minutes, the cells were washed and anti-laminin conjugated to AF650 (NovusBio) was added to detect the bound laminin. The cells were run on a flow cytometer to measure the laminin binding signal.

Table 6 provides the constructs used for transfection.

TABLE 6

| Construct | Description |
|---|---|
| h2120_VH1 | Standard |
| h2120_VH2 | Conservative |
| h2120_VH3 | Standard + N-S |
| h2120_VH4 | Standard + N-Q |
| h2120_VH5 | Standard + G-A |
| h2120_VL1 | Standard |
| h2120_VL2 | Aggressive |
| h2120_VL3 | Conservative |

Table 7 describes the specific transfection experiments.

TABLE 7

| | |
|---|---|
| Transfection--round 1 | |
| h2120_VH1 + h2120_VL3 | Standard VH + conservative VL |
| h2120_VH2 + h2120_VL3 | Conservative VH + conservative VL |
| h2120_VH3 + h2120_VL3 | N-S deamidate VH + conservative VL |
| Transfection--round 2 | |
| h2120_VH4 + h2120_VL3 | N-Q deamidate VH + conservative VL |
| h2120_VH5 + h2120_VL3 | G-A deamidate VH + conservative VL |
| h2120_VH1 + h2120_VL1 | Standard VH + standard VL |
| h2120_VH1 + h2120_VL2 | Standard VH + aggressive VL |

Table 8 shows the relative affinities of the humanized antibodies compared to the rodent parent as measured by ForteBio and competitive ELISA, as well as the expression levels for the first round of transfections.

TABLE 8

| | Forte | | ELISA | | |
|---|---|---|---|---|---|
| | Expt. #1 | Expt. #2 | Expt. #1 | Expt. #2 | |
| | Fold over | Fold over | Fold over | Fold over | Expression |
| Transfection--round 1 | rodent | rodent | rodent | rodent | level |
| rodent 2120 | 1.00 | 1.00 | 1.00 | 1.00 | |
| h2120_VH1 + h2120_VL3 | 5.64 | 6.21 | 2.23 | 2.42 | 22 mg/L |
| h2120_VH2 + h2120_VL3 | 6.57 | 6.43 | 1.93 | 2.62 | 16 mg/L |
| h2120_VH3 + h2120_VL3 | 16.14 | | 3.47 | | 22 mg/L |
| chimeric 2120 | | | 0.97 | 1.72 | |

Table 9 shows the measured affinity by ForteBio, competitive ELISA, and functional blocking data (laminin/FACS assay) compared to the rodent parent, as well as the expression levels, from the second round of transfections.

TABLE 9

| | | | | Blocking | | |
|---|---|---|---|---|---|---|
| | Forte | Forte | ELISA | Expt#1 | Expt#2 | |
| | Fold over | Fold over | Fold over | Fold over | Fold over | Expression |
| Transfection--round 2 | rodent | rodent | rodent | rodent | rodent | level |
| h2120_VH4 + h2120_VL3 | 17.4 | | 5.0 | 3.8 | 5.6 | 15 mgL |
| h2120_VH5 + h2120_VL3 | 1.1 | 1.2 | 2.4 | 1.2 | 1.5 | 22 mg/L |
| h2120_VH1 + h2120_VL1 | 8.8 | | 3.1 | 2.0 | 3.5 | 17 mg/L |
| h2120_VH1 + h2120_VL2 | 10.8 | | 3.1 | 4.6 | 12.6 | 2 mg/L |
| h2120_VH1 + h2120_VL3 | 5.9 | 5.8 | 1.8 | 1.7 | 2.8 | 22 mg/L |
| rodent 2120 | 1.0 | 1.0 | 1.0 | 1.0 | 1.0 | |

Overall, the data demonstrates that the various 2120 humanized antibodies have a >5× reduction in affinity as measured by ForteBio, and most have a >2-3× reduction in apparent affinity and potency as measured by the competitive ELISA and laminin blocking assay, with the exception of VH5VL3 (G-A N-deamidation mutant VH/conservative VL), which had a <2× reduction in affinity and potency.

Certain candidate antibodies were re-expressed and tested for their affinity by ForteBio and their IC₅₀. The results are provided in Table 10 below.

TABLE 10

| | | Blocking | |
|---|---|---|---|
| | Forte kD | IC50 | Expression |
| h2120VH5VL3 | 1.3 | 0.7 | 12.7 mg/L |

### Example 7. Modification of Humanized 2120 Antibodies

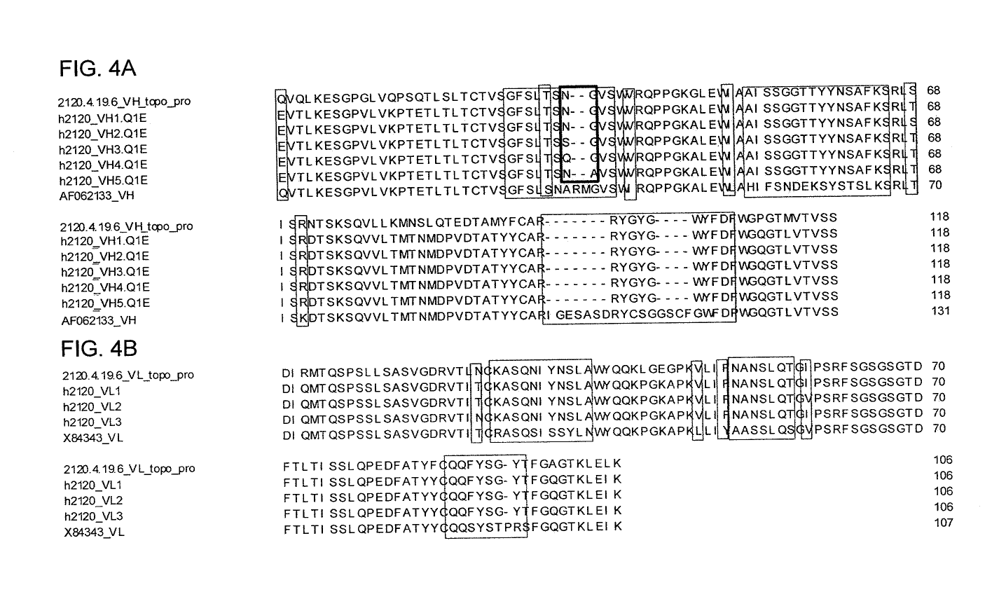

Utilizing the DNA manipulation methods described above and according to Liu et al. JBC. 286:11211-7, 2011, variants of the rat and humanized versions of the 2120.4.19 antibody mature heavy chain variable regions were constructed. Variants of 2120.4.19, h2120VH1, h2120VH2, h2120VH3, h2120VH4, and h2120VH5 were constructed having a glutamine to glutamic acid substitution at position H1 (Kabat numbering) (FIG. 4A). These variants are referred to as 2120.4.19.Q1E, h2120VH1.Q1E, h2120VH2.Q1E, h2120VH3.Q1E, h2120VH4.Q1E, and h2120VH5.Q1E and are shown in SEQ ID NOS:156-161. The humanized versions identified by SEQ ID NOs:157-161 are depicted in the alignment in FIG. 4A. Various rat and humanized antibodies can be constructed using the modified variable heavy chains, including: h2120VH1.Q1E+h2120VL1; h2120VH1.Q1E+h2120VL2; h2120VH1.Q1E+h2120VL3; h2120VH2.Q1E+h2120VL1; h2120VH2.Q1E+h2120VL2; h2120VH2.Q1E+h2120VL3; h2120VH3.Q1E+h2120VL1; h2120VH3.Q1E+h2120VL2; h2120VH3.Q1E+h2120VL3; h2120VH4.Q1E+h2120VL1; h2120VH4.Q1E+h2120VL2; h2120VH4.Q1E+h2120VL3; h2120VH5.Q1E+h2120VL1; h2120VH5.Q1E+h2120VL2; and h2120VH5.Q1E+h2120VL3.

1-4. (canceled)

5. The method of claim 77, wherein the mature heavy chain variable region is at least 90% identical to SEQ ID NO:161, and the mature light chain variable region is at least 90% identical to SEQ ID NO:123.

6. The method of claim 77, wherein the mature heavy chain variable region is at least 95% identical to SEQ ID NO:161 and the mature light chain variable region is at least 95% identical to SEQ ID NO:123.

7. The method of claim 77, wherein the mature heavy chain variable region is at least 98% identical to SEQ ID NO:161 and the mature light chain variable region is at least 95% identical to SEQ ID NO:123.

8. The method of claim 77, wherein the mature heavy chain variable region is at least 99% identical to SEQ ID NO:161 and the mature light chain variable region is at least 95% identical to SEQ ID NO:123.

9. The method of claim 77, wherein the mature heavy chain variable region has the amino acid sequence of SEQ ID NO:157, SEQ ID NO:158, SEQ ID NO:159, SEQ ID NO:160, or SEQ ID NO:161, and wherein the mature light chain variable region is at least 95% identical to SEQ ID NO:123.

10. The method of claim 77, wherein the mature heavy chain variable region is at least 95% identical to SEQ ID NO:161 and the mature light chain variable region is at least 98% identical to SEQ ID NO:123.

11. The method of claim 77, wherein the mature heavy chain variable region is at least 95% identical to SEQ ID NO:161 and the mature light chain variable region is at least 99% identical to SEQ ID NO:123.

12. The method of claim 77, wherein the mature heavy chain variable region is at least 95% identical to SEQ ID NO:161 and the mature light chain variable region has the amino acid sequence of SEQ ID NO:121, SEQ ID NO:122, or SEQ ID NO:123.

13. The method of claim 77, wherein the mature heavy chain variable region is at least 98% identical to SEQ ID NO:161 and the mature light chain variable region is at least 98% identical to SEQ ID NO:123.

14. The method of claim 77, wherein the mature heavy chain variable region is at least 99% identical to SEQ ID NO:161 and the mature light chain variable region is at least 99% identical to SEQ ID NO:123.

15. The method of claim 77, wherein the mature heavy chain variable region has the amino acid sequence of SEQ ID NO:157, SEQ ID NO:158, SEQ ID NO:159, SEQ ID NO:160, or SEQ ID NO:161, and wherein the mature light chain variable region has the amino acid sequence of SEQ ID NO:121, SEQ ID NO:122, or SEQ ID NO:123.

16. The method of claim 77, wherein the mature heavy chain variable region has the amino acid sequence of SEQ ID NO:161, and wherein the mature light chain variable region has the amino acid sequence of SEQ ID NO:123.

17. The method of claim 77, wherein the mature heavy chain variable region has the amino acid sequence of SEQ ID NO:161 and the mature light chain variable region has the amino acid sequence of SEQ ID NO:123, and wherein the antibody comprises a heavy chain constant region having the amino acid sequence of SEQ ID NO:171 and a light chain constant region having the amino acid sequence of SEQ ID NO:168.

18. The method of claim 77, wherein the mature heavy chain variable region has the amino acid sequence of SEQ ID NO:161 and the mature light chain variable region has the amino acid sequence of SEQ ID NO:123, and wherein the antibody comprises a heavy chain constant region having the amino acid sequence of SEQ ID NO:172 and a light chain constant region having the amino acid sequence of SEQ ID NO:168.

19. The method of claim 1, wherein the antibody is a humanized antibody.

20-30. (canceled)

31. A method for treating or effecting prophylaxis of giant cell arteritis, polymyalgia rheumatic (PMR) or Takayasu's arteritis, the method comprising administering to a mammalian subject in need thereof an effective amount of a pharmaceutical formulation comprising:
(a) an antibody comprising:
(i) a mature heavy chain variable region comprising the three Kabat CDRs of SEQ ID NO:161 except that position 32 (Kabat numbering) can be N, S, or Q, and position 33 (Kabat numbering) can be G or A; and
(ii) a mature light chain variable region comprising the three Kabat CDRs of SEQ ID NO:123;
(b) histidine buffer present at a concentration within the range from about 10 mM to about 30 mM;
(c) one or more sugars and polyols (“sugar/polyol”) selected from:
(i) sucrose present at a concentration within the range from about 200 mM to about 260 mM; and
(ii) trehalose present at a concentration within the range from about 200 mM to about 260 mM; and
(d) polysorbate 20 present at a concentration within the range from about 0.005% to about 0.05% by weight;
wherein the pharmaceutical formulation is characterized by a pH within the range from about 5.5 to about 7.

32. The method of claim 31 wherein the pharmaceutical formulation is administered for the treatment or prophylaxis of giant cell arteritis.

33. The method of claim 31 wherein the pharmaceutical formulation is administered for the treatment or prophylaxis of PMR.

34. The method of claim 31 wherein the pharmaceutical formulation is administered for the treatment or prophylaxis of Takayasu's arteritis.

35. The method of claim 31, wherein the mature heavy chain variable region is at least 90% identical to SEQ ID NO:161, and the mature light chain variable region is at least 90% identical to SEQ ID NO:123.

36. The method of claim 31, wherein position 1 (Kabat numbering) of the mature heavy chain variable region is occupied by E.

37. The method of claim 31, wherein the mature heavy chain variable region has the amino acid sequence of SEQ ID NO:157, SEQ ID NO:158, SEQ ID NO:159, SEQ ID NO:160, or SEQ ID NO:161, and wherein the mature light chain variable region has the amino acid sequence of SEQ ID NO:121, SEQ ID NO:122, or SEQ ID NO:123.

38. The method of claim 31, wherein the mature heavy chain variable region has the amino acid sequence of SEQ ID NO:161 and the mature light chain variable region has the amino acid sequence of SEQ ID NO:123.

39. The method of claim 31, wherein the mature heavy chain variable region has the amino acid sequence of SEQ ID NO:161 and the mature light chain variable region has the amino acid sequence of SEQ ID NO:123, and wherein the antibody comprises a heavy chain constant region having the amino acid sequence of SEQ ID NO:171 and a light chain constant region having the amino acid sequence of SEQ ID NO:168.

40. The method of claim 31, wherein the mature heavy chain variable region has the amino acid sequence of SEQ ID NO:161 and the mature light chain variable region has the amino acid sequence of SEQ ID NO:123, and wherein the antibody comprises a heavy chain constant region having the amino acid sequence of SEQ ID NO:172 and a light chain constant region having the amino acid sequence of SEQ ID NO:168.

41. A method for treating or effecting prophylaxis of giant cell arteritis, polymyalgia rheumatic (PMR) or Takayasu's arteritis, the method comprising administering to a mammalian subject in need thereof an effective amount of a pharmaceutical formulation comprising:
(a) an antibody that binds to human MCAM (SEQ ID NO:11) at an epitope including amino acid residue 141;
(b) histidine buffer present at a concentration within the range from about 10 mM to about 30 mM;
(c) one or more sugars and polyols (“sugar/polyol”) selected from:
(i) sucrose present at a concentration within the range from about 200 mM to about 260 mM; and
(ii) trehalose present at a concentration within the range from about 200 mM to about 260 mM; and
(d) polysorbate 20 present at a concentration within the range from about 0.005% to about 0.05% by weight;
wherein the pharmaceutical formulation is characterized by a pH within the range from about 5.5 to about 7 for use in treatment or prophylaxis of giant cell arteritis, polymyalgia rheumatica (PMR) or Takayasu's arteritis.

42. (canceled)

43. The method of claim 31 or 41, wherein the antibody is present at a concentration of about 40 mg/mL.

44. The method of claim 31 or 41, wherein the histidine buffer is present at a concentration of about 20 mM.

45. The method of claim 31 or 41, wherein the sugar/polyol is sucrose present at a concentration of about 220 mM.

46. The method of claim 31 or 41, wherein the pH is about 6.0.

47. The method of claim 31 or 41, which is characterized by an osmolality of about 295 mOsm/kg.

48. The method of claim 31 or 41, wherein the sugar/polyol is trehalose present at a concentration of about 220 mM.

49. The method of claim 31 or 41, wherein the pH is about 6.5.

50. The method of claim 31 or 41, which is characterized by an osmolality of about 287 mOsm/kg.

51. The method of claim 31 or 41, wherein less than about 5% of the antibody or the isolated anti-MCAM antibody is present as an aggregate in the formulation.

52-53. (canceled)

54. The method of claim 31 or 41, which is stable on freezing and thawing.

55. The method of claim 31 or 41, in which at least 65% of protein appears as a single peak on hydrophobic interaction chromatography after storage for at least 30 days at 38-42° C. and/or after storage for at least 3 months at 38-42° C.

56. The method of claim 31 or 41, having no more than 5% aggregated protein by weight on high performance size exclusion chromatography after storage for at least 30 days at 38-42° C. and/or after storage for at least 3 months at 38-42° C.

57-76. (canceled)

77. A method for treating or effecting prophylaxis of giant cell arteritis, polymyalgia rheumatica (PMR) or Takayasu's arteritis, the method comprising administering to a mammalian subject in need thereof an effective amount of an antibody comprising:
(a) a mature heavy chain variable region comprising the three Kabat CDRs of SEQ ID NO:161 except that position 32 (Kabat numbering) can be N, S, or Q, and position 33 (Kabat numbering) can be G or A, and wherein position 1 (Kabat numbering) is occupied by E; and
(b) a mature light chain variable region comprising the three Kabat CDRs of SEQ ID NO:123.

78. The method of claim 77, wherein the MCAM-expressing cells are TH17 cells.

79. The method of any of claims 31, 41, 77, 80, 83 or 89, wherein the mammalian subject is a human.

80. A method for treating or effecting prophylaxis of giant cell arteritis, polymyalgia rheumatica (PMR) or Takayasu's arteritis, the method comprising administering to a mammalian subject in need thereof an effective amount of an isolated peptide comprising 5-50 contiguous amino acid residues of human MCAM (SEQ ID NO:11) including amino acid residue 141.

81. The method of claim 80, wherein the peptide is linked to a carrier polypeptide.

82. The method of claim 80, wherein the peptide is combined with an adjuvant.

83. A method for treating or effecting prophylaxis of giant cell arteritis, polymyalgia rheumatica (PMR) or Takayasu's arteritis, the method comprising administering to a mammalian subject in need thereof an effective amount of a humanized 2107 antibody.

84. The method of claim 83, wherein the antibody comprises a mature heavy chain variable region of SEQ ID NO:178 or 179.

85. The method of claim 83 or 84, wherein the antibody further comprises a mature light chain variable region of SEQ ID NO:98.

86. The method of claim 77, wherein the antibody is administered for the treatment or prophylaxis of giant cell arteritis.

87. The method of claim 77, wherein the antibody is administered for the treatment or prophylaxis of PMR.

88. The method of claim 77, wherein the antibody is administered for the treatment or prophylaxis of Takayasu's arteritis.

89. A method for treating or effecting prophylaxis of giant cell arteritis, polymyalgia rheumatica (PMR) or Takayasu's arteritis, the method comprising administering to a mammalian subject in need thereof an effective amount of an antibody that binds to human MCAM (SEQ ID NO:11) at an epitope including amino acid residue 141 for use in treatment or prophylaxis of giant cell arteritis.

90. The method of claim 89, wherein the antibody is administered for the treatment or prophylaxis of giant cell arteritis.

91. The method of claim 89, wherein the antibody is administered for the treatment or prophylaxis of PMR.

92. The method of claim 89, wherein the antibody is administered for the treatment or prophylaxis of Takayasu's arteritis.

93. The method of claim 89, wherein the epitope comprises amino acid residue 145.

94. The method of any one of claims 89-93, wherein the antibody is not monoclonal antibody 2120.4.19 or an antibody comprising CDRs substantially from monoclonal antibody 2120.4.19.

95. The method of claim 89, wherein the antibody is monoclonal.

96. The method of claim 95, wherein the antibody is chimeric, humanized, veneered or human.


SEQUENCE LISTING (179 sequences)

SEQ ID NO: 1 - DNA, 428 bp, Artificial Sequence
  Synthetic
atgagggtccagattcagtttctggggctccttctgctctggacatcagttgtccagtgtgatgtccagatgacccagtctccatcttatcttgctacgtctcctggagagagtgtttccatcagttgcaaggcaagtaaaaacattgacacatacttagcctggtatcaggagaaacctgggaaaacgaataagcttcttatctactctgggtcaactttgcaatctggaactccatcgagattcagtggcagtggatctggtacagatttcacgctcaccatcagaaacctggagtctgaagattttgcagtctactactgtcaacagcataatgaatacccgctcacgttcggttctgggaccaagctggagatcaaacgggctgatgctgcaccaactgtatccatcttcccaccatcctcgga

SEQ ID NO: 2 - protein, 142 aa, Artificial Sequence
  Synthetic
MRVQIQFLGLLLLWTSVVQCDVQMTQSPSYLATSPGESVSISCKASKNIDTYLAWYQEKPGKTNKLLIYSGSTLQSGTPSRFSGSGSGTDFTLTIRNLESEDFAVYYCQQHNEYPLTFGSGTKLEIKRADAAPTVSIFPPSS

SEQ ID NO: 3 - protein, 11 aa, Artificial Sequence
  Synthetic
KASKNIDTYLA

SEQ ID NO: 4 - protein, 5 aa, Artificial Sequence
  Synthetic
SGSTL

SEQ ID NO: 5 - protein, 9 aa, Artificial Sequence
  Synthetic
QQHNEYPLT

SEQ ID NO: 6 - DNA, 480 bp, Artificial Sequence
  Synthetic
atggacaccaggctctgcttggttttccttgtccttttcataaaaggtgtccagtgtgaggtgcagctggtggagtctggtggaggcttagtgcagcctggaaggtccctgaaactctcctgtgcagcctcaggattcactttcagtaactattacatggcctgggtccgccaggctccaacgaagggtctggagtgggtcgcatccattagttttgagggtaatagaaatcactatggagactccgtgaagggccgaatcactatctccagagataatgcaaaaagcaccctatacctgcaaatgaccagtctgaggcctgaggacacggcctattattgtgcaagacatcgggggtatagtacgaatttttatcacgacgttttggatgcctggggtcaaggagctttagtcactgtctcctcagctgaaacaacagccccatctgtctatccactggctcctggaactgctctcaaa

SEQ ID NO: 7 - protein, 161 aa, Artificial Sequence
  Synthesized
MDTRLCLVFLVLFIKGVQCEVQLVESGGGLVQPGRSLKLSCAASGFTFSNYYMAWVRQAPTKGLEWVASISFEGNRNHYGDSVKGRITISRDNAKSTLYLQMTSLRPEDTATYYCARHRGYSTNFYHDVLDAWGQGALVTVSSAETTAPSVYPLAPGTALK

SEQ ID NO: 8 - protein, 10 aa, Artificial Sequence
  Synthetic
GFTFSNYYMA

SEQ ID NO: 9 - protein, 16 aa, Artificial Sequence
  Synthetic
SISFEGNRNHYGDSVK

SEQ ID NO: 10 - protein, 19 aa, Artificial Sequence
  Synthetic
HRGYSTNFYHDVLDAWGQG

SEQ ID NO: 11 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKTELVVEVKSDKLPEEMGLLQGSSGDKRAPGDQGEKYIDLRH

SEQ ID NO: 12 - DNA, 474 bp, Artificial Sequence
  Synthetic
atggaatcacagacccaggtcctcatgtccctgctgctctggatttctggtacctgtggggacattgtgatgacccagtctccatcctctctggctgtgtcagctggggagacggtctctatacactgcaagtccagtcagagtcttttatacagtggaacccaaaagaactacttggcctggttccagcagaaaccaggacagtctcctaaactgctgatcttctgggcatctactaggcagtctggtgtccctgatcgcttcataggccgtggatctgggacagacttcactctgaccatcagcggtgtgcaggcagaagatctggcaatttattactgtcaacaatattatgatactctcacggacacgtttggagcggggaccaagctggaactgaaacgggctgatgctgcaccaactgtatctatcttcccaccatccacggaacagttagcaactggaggtgcctca

SEQ ID NO: 13 - protein, 158 aa, Artificial Sequence
  Synthesized
MESQTQVLMSLLLWISGTCGDIVMTQSPSSLAVSAGETVSIHCKSSQSLLYSGTQKNYLAWFQQKPGQSPKLLIFWASTRQSGVPDRFIGRGSGTDFTLTISGVQAEDLAIYYCQQYYDTLTDTFGAGTKLELKRADAAPTVSIFPPSTEQLATGGAS

SEQ ID NO: 14 - protein, 17 aa, Artificial Sequence
  Synthetic
KSSQSLLYSGTQKNYLA

SEQ ID NO: 15 - protein, 7 aa, Artificial Sequence
  Synthetic
WASTRQS

SEQ ID NO: 16 - protein, 10 aa, Artificial Sequence
  Synthetic
QQYYDTLTDT

SEQ ID NO: 17 - DNA, 469 bp, Artificial Sequence
  Synthetic
atggacatcaggctcagcttggctttcctggtccttttcataaaaggtgtccagtgtgaggtgcggctggtggagtctgggggaggcttagtgcagcctggaaagtccatgaaactctcctgtgtagcctcgggattcaaattcagtaactattacatgtcctgggtccgccaggctccagcgaagggtctggagtgggtcgcatccattagtgatggtggtggtgacactttctgtcgagacttggtgaagggccgattcactatctccagagataatgcaaaaagtaccctttacctgcaaatggacagtctgaggcctgaggacacggccacttattactgtgcaagacggggagcagctatggggggtgttatggatgcctggggtcaaggaacttcagtcactgtctcctcagctgaaacaacagccccatctgtctatccactggctcctggaactgctctca

SEQ ID NO: 18 - protein, 156 aa, Artificial Sequence
  Synthesized
MDIRLSLAFLVLFIKGVQCEVRLVESGGGLVQPGKSMKLSCVASGFKFSNYYMSWVRQAPAKGLEWVASISDGGGDTFCRDLVKGRFTISRDNAKSTLYLQMDSLRPEDTATYYCARRGAAMGGVMDAWGQGTSVTVSSAETTAPSVYPLAPGTAL

SEQ ID NO: 19 - protein, 10 aa, Artificial Sequence
  Synthetic
GFKFSNYYMS

SEQ ID NO: 20 - protein, 17 aa, Artificial Sequence
  Synthetic
SISDGGGDTFCRDLVKG

SEQ ID NO: 21 - protein, 15 aa, Artificial Sequence
  Synthetic
RGAAMGGVMDAWGQG

SEQ ID NO: 22 - protein, 111 aa, Homo sapiens
PRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQ

SEQ ID NO: 23 - protein, 104 aa, Homo sapiens
PNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVT

SEQ ID NO: 24 - protein, 78 aa, Homo sapiens
PVFYPTEKVWLEVEPVGMLKEGDRVEIRCLADGNPPPHFSISKQNPSTREAEEETTNDNGVLVLEPARKEHSGRYECQ

SEQ ID NO: 25 - protein, 90 aa, Homo sapiens
PQELLVNYVSDVRVSPAAPERQEGSSLTLTCEAESSQDLEFQWLREETDQVLERGPVLQLHDLKREAGGGYRCVASVPSIPGLNRTQLVK

SEQ ID NO: 26 - protein, 81 aa, Homo sapiens
PPWMAFKERKVWVKENMVLNLSCEASGHPRPTISWNVNGTASEQDQDPQRVLSTLNVLVTPELLETGVECTASNDLGKNTS

SEQ ID NO: 27 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 28 - protein, 1816 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 29 - DNA, 334 bp, Artificial Sequence
  Synthetic
gacattgtgctgacacagtctcctgcttccttagctgtatctctggggcagagggccaccatctcatgcagggccagcaaaagtgtcagtacatctggctatagttatatgtactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcaacacagtagggagcttccattcacgttcggctcggggacaaagttggaaataaaac

SEQ ID NO: 30 - protein, 111 aa, Artificial Sequence
  Synthesized
DIVLTQSPASLAVSLGQRATISCRASKSVSTSGYSYMYWYQQKPGQPPKLLIYLASNLESGVPARFSGSGSGTDFTLNIHPVEEEDAATYYCQHSRELPFTFGSGTKLEIK

SEQ ID NO: 31 - protein, 15 aa, Artificial Sequence
  Synthetic
RASKSVSTSGYSYMY

SEQ ID NO: 32 - protein, 6 aa, Artificial Sequence
  Synthetic
ASNLES

SEQ ID NO: 33 - protein, 9 aa, Artificial Sequence
  Synthetic
QHSRELPFT

SEQ ID NO: 34 - DNA, 363 bp, Artificial Sequence
  Synthetic
cagattcagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttgtctttggaaacctctgccagcactgcctatttgcagatcaacaacctcaaaaatgaggacatggctacatatttctgtgcaagatataggtataataaatacgagagggctatggactactggggtcaaggaacctcagtcaccgtctcctca

SEQ ID NO: 35 - protein, 121 aa, Artificial Sequence
  Synthesized
QIQLVQSGPELKKPGETVKISCKASGYTFTNYGMNWVKQAPGKGLKWMGWINTYTGEPTYADDFKGRFALSLETSASTAYLQINNLKNEDMATYFCARYRYNKYERAMDYWGQGTSVTVSS

SEQ ID NO: 36 - protein, 10 aa, Artificial Sequence
  Synthetic
GYTFTNYGMN

SEQ ID NO: 37 - protein, 17 aa, Artificial Sequence
  Synthetic
WINTYTGEPTYADDFKG

SEQ ID NO: 38 - protein, 12 aa, Artificial Sequence
  Synthetic
YRYNKYERAMDY

SEQ ID NO: 39 - DNA, 338 bp, Artificial Sequence
  Synthetic
gacattgtgatgtcacagtctccatcctccctggctgtgtcagcaggagagaaggtcactatgagctgcaaatccagtcagagtctgctcaacagtagcacccgaaagaacttcttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatcggtacacgttcggaggggggaccaagctggaaataaaacg

SEQ ID NO: 40 - protein, 112 aa, Artificial Sequence
  Synthesized
DIVMSQSPSSLAVSAGEKVTMSCKSSQSLLNSSTRKNFLAWYQQKPGQSPKLLIYWASTRESGVPDRFTGSGSGTDFTLTISSVQAEDLAVYYCKQSYNRYTFGGGTKLEIK

SEQ ID NO: 41 - protein, 17 aa, Artificial Sequence
  Synthetic
KSSQSLLNSSTRKNFLA

SEQ ID NO: 42 - protein, 7 aa, Artificial Sequence
  Synthetic
WASTRES

SEQ ID NO: 43 - protein, 8 aa, Artificial Sequence
  Synthetic
KQSYNRYT

SEQ ID NO: 44 - DNA, 351 bp, Artificial Sequence
  Synthetic
gagatccagctgcagcagactggacctgagctggtgaagcctggggcttcagtgaagatatcctgcaaggcttctggttattcattcactgactacatcatgctctgggtgaagcagagccatggaaagagccttgagtggattggaaatattaatccttactctggtagtagtggctacaatctgaagttcaagggcaaggccacattgactgtagacaaatcttccagcacagcctacatgcagctcaacagtctgacatctgaggactctgcagtctattactgtgcaagagggaaggactttgctatggactactggggtcaaggaacctcagtcaccgtctcctca

SEQ ID NO: 45 - protein, 117 aa, Artificial Sequence
  Synthesized
EIQLQQTGPELVKPGASVKISCKASGYSFTDYIMLWVKQSHGKSLEWIGNINPYSGSSGYNLKFKGKATLTVDKSSSTAYMQLNSLTSEDSAVYYCARGKDFAMDYWGQGTSVTVSS

SEQ ID NO: 46 - protein, 10 aa, Artificial Sequence
  Synthetic
GYSFTDYIML

SEQ ID NO: 47 - protein, 17 aa, Artificial Sequence
  Synthetic
NINPYSGSSGYNLKFKG

SEQ ID NO: 48 - protein, 7 aa, Artificial Sequence
  Synthetic
GKDFAMD

SEQ ID NO: 49 - DNA, 322 bp, Artificial Sequence
  Synthetic
gacattgtgatgactcagtctccagccaccctgtctgtgactccaggagatagagtctctctttcatgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacaactttcctcggacgttcggtggaggcaccaagctggaaatcaaac

SEQ ID NO: 50 - protein, 107 aa, Artificial Sequence
  Synthesized
DIVMTQSPATLSVTPGDRVSLSCRASQSISDYLHWYQQKSHESPRLLIKYASQSISGIPSRFSGSGSGSDFTLSINSVEPEDVGVYYCQNGHNFPRTFGGGTKLEIK

SEQ ID NO: 51 - protein, 11 aa, Artificial Sequence
  Synthetic
RASQSISDYLH

SEQ ID NO: 52 - protein, 7 aa, Artificial Sequence
  Synthetic
YASQSIS

SEQ ID NO: 53 - protein, 9 aa, Artificial Sequence
  Synthetic
QNGHNFPRT

SEQ ID NO: 54 - DNA, 366 bp, Artificial Sequence
  Synthetic
caggtccaactgcagcagcctggggctgagcttgtgcagcctggggctccagtgaagctgtcctgcaaggcttctggctacattttcaccagctactggatgaactgggtgaagcagaggcctggacgaggcctcgagtggattggaaggattgatccttccgatagtaaaattcactacaatcaaaagttcaaagacaaggccacactgactgtagacagatcctccagcacagcctacatccaactcggcagcctgacatctgaggactctgcggtctattattgtgcaaaagaggggggtttacgacggggggactatgctatggactactggggtcaaggaacctcagtcaccgtctcctca

SEQ ID NO: 55 - protein, 122 aa, Artificial Sequence
  Synthesized
QVQLQQPGAELVQPGAPVKLSCKASGYIFTSYWMNWVKQRPGRGLEWIGRIDPSDSKIHYNQKFKDKATLTVDRSSSTAYIQLGSLTSEDSAVYYCAKEGGLRRGDYAMDYWGQGTSVTVSS

SEQ ID NO: 56 - protein, 10 aa, Artificial Sequence
  Synthetic
GYIFTSYWMN

SEQ ID NO: 57 - protein, 17 aa, Artificial Sequence
  Synthetic
RIDPSDSKIHYNQKFKD

SEQ ID NO: 58 - protein, 13 aa, Artificial Sequence
  Synthetic
EGGLRRGDYAMDY

SEQ ID NO: 59 - DNA, 337 bp, Artificial Sequence
  Synthetic
gacattgtgatgtcacagtctccatcctccctggctgtgtcagcaggagagaaggtcactatgaactgcaaatccagtcggagtctgctcaacagtagaatccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgctcacgttcggtgctgggaccaagctggagctgaaac

SEQ ID NO: 60 - protein, 112 aa, Artificial Sequence
  Synthesized
DIVMSQSPSSLAVSAGEKVTMNCKSSRSLLNSRIRKNYLAWYQQKPGQSPKLLIYWASTRESGVPDRFTGSGSGTDFTLTISSVQAEDLAVYYCKQSYNLLTFGAGTKLELK

SEQ ID NO: 61 - protein, 17 aa, Artificial Sequence
  Synthetic
KSSRSLLNSRIRKNYLA

SEQ ID NO: 62 - protein, 7 aa, Artificial Sequence
  Synthetic
WASTRES

SEQ ID NO: 63 - protein, 8 aa, Artificial Sequence
  Synthetic
KQSYNLLT

SEQ ID NO: 64 - DNA, 360 bp, Artificial Sequence
  Synthetic
gacgtgaagctggtggagtctgggggagacttagtgaagcctggagggtccctgaaactctcctgtgcagcctctggattcactttcagtagctatatcatgtcttgggttcgtcagactccggagaagaggctggagtgggtcgcaaccattagtagtggtggtagttccacctactatccagacagtgtgaagggccgattcaccatctccagagacaatgccaagaacaccctgtacctgcaaatgagcagtctgaagtctgaggacacagccatgtattactgtacaagagatgatgattacgacgtaaaggtatttgcttactggggccaagggactctggtcactgtctctgca

SEQ ID NO: 65 - protein, 120 aa, Artificial Sequence
  Synthesized
DVKLVESGGDLVKPGGSLKLSCAASGFTFSSYIMSWVRQTPEKRLEWVATISSGGSSTYYPDSVKGRFTISRDNAKNTLYLQMSSLKSEDTAMYYCTRDDDYDVKVFAYWGQGTLVTVSA

SEQ ID NO: 66 - protein, 5 aa, Artificial Sequence
  Synthetic
SYIMS

SEQ ID NO: 67 - protein, 17 aa, Artificial Sequence
  Synthetic
TISSGGSSTYYPDSVKG

SEQ ID NO: 68 - protein, 11 aa, Artificial Sequence
  Synthetic
DDDYDVKVFAY

SEQ ID NO: 69 - DNA, 319 bp, Artificial Sequence
  Synthetic
gatatccggatgactcagtctccttcactcctgtctgcatctgtgggggacagagtcactctcaactgcaaagcaagtcagaatatttataacagcttagcctggtatcagcaaaagcttggagaaggtcccaaagtcctgatttttaatgcaaacagtttgcaaacgggcatcccatcaaggttcagtggcagtggatctggtacagatttcacactcaccatcagcagcctgcagcctgaagattttgccacatatttctgccagcagttttatagcgggtacacgtttggagctgggaccaagctggaactgaaac

SEQ ID NO: 70 - protein, 106 aa, Artificial Sequence
  Synthesized
DIRMTQSPSLLSASVGDRVTLNCKASQNIYNSLAWYQQKLGEGPKVLIFNANSLQTGIPSRFSGSGSGTDFTLTISSLQPEDFATYFCQQFYSGYTFGAGTKLELK

SEQ ID NO: 71 - protein, 106 aa, Artificial Sequence
  Synthetic
DIQVTQSPSLLSASVGDRVTLNCKASQNIYNSLAWYQQKLGEGPKVLIFNANSLQTGIPSRFSGSGSGTDFTLTISSLQPEDFATYFCQQFYSGYTFGAGTKLELK

SEQ ID NO: 72 - protein, 106 aa, Artificial Sequence
  Synthetic
DIVLTQSPSLLSASVGDRVTLNCKASQNIYNSLAWYQQKLGEGPKVLIFNANSLQTGIPSRFSGSGSGTDFTLTISSLQPEDFATYFCQQFYSGYTFGAGTKLELK

SEQ ID NO: 73 - protein, 11 aa, Artificial Sequence
  Synthetic
KASQNIYNSLA

SEQ ID NO: 74 - protein, 7 aa, Artificial Sequence
  Synthetic
NANSLQT

SEQ ID NO: 75 - protein, 8 aa, Artificial Sequence
  Synthetic
QQFYSGYT

SEQ ID NO: 76 - DNA, 354 bp, Artificial Sequence
  Synthetic
caggtgcagctgaaggagtcaggacctggtctggtgcagccctcacagaccctgtctctcacctgcactgtctctggattctcattaaccagcaatggtgtaagctgggttcgccagcctccaggaaagggtctggagtggattgcagcaatatcatctggtggaaccacatattataattcagcgttcaaatcccgactgagcatcagcaggaacacctccaagagccaagttctcttaaaaatgaacagtctgcaaactgaagacacagccatgtacttctgtgccagacggtatgggtacgggtggtactttgacttctggggcccaggaaccatggtcacagtctcctca

SEQ ID NO: 77 - protein, 118 aa, Artificial Sequence
  Synthesized
QVQLKESGPGLVQPSQTLSLTCTVSGFSLTSNGVSWVRQPPGKGLEWIAAISSGGTTYYNSAFKSRLSISRNTSKSQVLLKMNSLQTEDTAMYFCARRYGYGWYFDFWGPGTMVTVSS

SEQ ID NO: 78 - protein, 10 aa, Artificial Sequence
  Synthetic
GFSLTSNGVS

SEQ ID NO: 79 - protein, 16 aa, Artificial Sequence
  Synthetic
AISSGGTTYYNSAFKS

SEQ ID NO: 80 - protein, 10 aa, Artificial Sequence
  Synthetic
RYGYGWYFDF

SEQ ID NO: 81 - DNA, 318 bp, Artificial Sequence
  Synthetic
gacatccgggtgactcagtctccttcactcctgtctgcatctgtgggagacagagtcactctcaactgcaaaggaagtcagaatatttataagagcttagcctggtttcggctaaagcgtggagaagctcccaagctcctgatttatgatgcaaacagtttgcaaacgggcatcccatcaaggttcagtggcagtggatctggtacagatttcacactcaccatcaccagcctacagcctgaagatgttgccacatatttctgccagcagtattatagcggttacacgtttggagctgggaccaagctggaactgaaa

SEQ ID NO: 82 - protein, 106 aa, Artificial Sequence
  Synthesized
DIRVTQSPSLLSASVGDRVTLNCKGSQNIYKSLAWFRLKRGEAPKLLIYDANSLQTGIPSRFSGSGSGTDFTLTITSLQPEDVATYFCQQYYSGYTFGAGTKLELK

SEQ ID NO: 83 - DNA, 318 bp, Artificial Sequence
  Synthetic
gacatccaggtgactcagtctccttcactcctgtctgcatctgtgggagacagagtcactctcaactgcaaaggaagtcagaatatttataagagcttagcctggtttcggctaaagcgtggagaagctcccaagctcctgatttatgatgcaaacagtttgcaaacgggcatcccatcaaggttcagtggcagtggatctggtacagatttcacactcaccatcaccagcctacagcctgaagatgttgccacatatttctgccagcagtattatagcggttacacgtttggagctgggaccaagctggaactgaaa

SEQ ID NO: 84 - protein, 106 aa, Artificial Sequence
  Synthesized
DIQVTQSPSLLSASVGDRVTLNCKGSQNIYKSLAWFRLKRGEAPKLLIYDANSLQTGIPSRFSGSGSGTDFTLTITSLQPEDVATYFCQQYYSGYTFGAGTKLELK

SEQ ID NO: 85 - protein, 11 aa, Artificial Sequence
  Synthetic
KGSQNIYKSLA

SEQ ID NO: 86 - protein, 7 aa, Artificial Sequence
  Synthetic
DANSLQT

SEQ ID NO: 87 - protein, 8 aa, Artificial Sequence
  Synthetic
QQYYSGYT

SEQ ID NO: 88 - DNA, 348 bp, Artificial Sequence
  Synthetic
caggtgcagctgaaggagtcaggacctggtctggtgcagtcctcacagaccctgtctctcacctgcactgtctctggattctcattaaccagtaatggtgtaagctgggttcgccagcctccaggaaagggtctggagtggattgcagcaatatcaagtggtggaagcacatattataattcagcgttcaaatcccgactgagcatcagcaggaacacctccaagagccaagttctcttaaaaatgaacagtctgcaaactgaagacacaggcatgtacttctgtgccagacatagaccgttctactttgattactggggccaaggagtcatggtcacagtctcctca

SEQ ID NO: 89 - protein, 116 aa, Artificial Sequence
  Synthesized
QVQLKESGPGLVQSSQTLSLTCTVSGFSLTSNGVSWVRQPPGKGLEWIAAISSGGSTYYNSAFKSRLSISRNTSKSQVLLKMNSLQTEDTGMYFCARHRPFYFDYWGQGVMVTVSS

SEQ ID NO: 90 - protein, 10 aa, Artificial Sequence
  Synthetic
GFSLTSNGVS

SEQ ID NO: 91 - protein, 16 aa, Artificial Sequence
  Synthetic
AISSGGSTYYNSAFKS

SEQ ID NO: 92 - protein, 8 aa, Artificial Sequence
  Synthetic
HRPFYFDY

SEQ ID NO: 93 - protein, 120 aa, Artificial Sequence; identical to SEQ ID NO: 65
  Synthetic

SEQ ID NO: 94 - protein, 120 aa, Artificial Sequence
  Synthetic
EVQLVESGGGLVQPGGSLRLSCAASGFTFSSYIMSWVRQAPGKGLEWVATISSGGSSTYYPDSVKGRFTISRDNAKNSLYLQMNSLRAEDTAVYYCTRDDDYDVKVFAYWGQGTMVTVSS

SEQ ID NO: 95 - protein, 120 aa, Artificial Sequence
  Synthetic
EVQLVESGGGLVQPGGSLRLSCAASGFTFSSYIMSWVRQAPGKRLEWVATISSGGSSTYYPDSVKGRFTISRDNAKNSLYLQMNSLRAEDTAVYYCTRDDDYDVKVFAYWGQGTMVTVSS

SEQ ID NO: 96 - protein, 120 aa, Artificial Sequence
  Synthetic
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDGAIFGVVSHIWGQGTMVTVSS

SEQ ID NO: 97 - protein, 112 aa, Artificial Sequence; identical to SEQ ID NO: 60
  Synthetic

SEQ ID NO: 98 - protein, 112 aa, Artificial Sequence
  Synthetic
DIVMTQSPDSLAVSLGERATINCKSSRSLLNSRIRKNYLAWYQQKPGQPPKLLIYWASTRESGVPDRFSGSGSGTDFTLTISSLQAEDVAVYYCKQSYNLLTFGQGTKVEIK

SEQ ID NO: 99 - protein, 112 aa, Artificial Sequence
  Synthetic
DIVMTQSPDSLAVSLGERATINCKSSRSLLNSRIRKNYLAWYQQKPGQPPKLLIYWASTRESGVPDRFTGSGSGTDFTLTISSLQAEDVAVYYCKQSYNLLTFGQGTKVEIK

SEQ ID NO: 100 - protein, 113 aa, Artificial Sequence
  Synthetic
DIVMTQSPDSLAVSLGERATINCKSSQSILYSSDNKNYLAWYQQKPGQPPKLLIYWASTRESGVPDRFSGSGSGTDFTLTISSLQAEDVAVYYCQQYYNLPWTFGQGTKVEIK

SEQ ID NO: 101 - protein, 116 aa, Artificial Sequence
  Synthetic
QVQLKESGPGLVQSSQTLSLTCTVSGFSLTSNGVSWVRQPPGKGLEWIAAISSGGSTYYNSAFKSRLSISPNTSKSQVLLKMNSLQTEDTGMYFCARHRPFYFDYWGQGVMVTVSS

SEQ ID NO: 102 - protein, 116 aa, Artificial Sequence
  Synthetic
QVTLKESGPVLVKPTETLTLTCTVSGFSLTSNGVSWVRQPPGKALEWIAAISSGGSTYYNSAFKSRLTISRDTSKSQVVLTMTNMDPVDTATYYCARHRPFYFDYWGQGTLVTVSS

SEQ ID NO: 103 - protein, 116 aa, Artificial Sequence
  Synthetic
QVTLKESGPVLVKPTETLTLTCTVSGFSLTSNGVSWVRQPPGKALEWIAAISSGGSTYYNSAFKSRLSISRDTSKSQVVLTMTNMDPVDTATYYCARHRPFYFDYWGQGTLVTVSS

SEQ ID NO: 104 - protein, 116 aa, Artificial Sequence
  Synthetic
QVTLKESGPVLVKPTETLTLTCTVSGFSLTSNGVSWVRQPPGKALEWIAAISSGGSTYYNSAFKSRLTISRNTSKSQVVLTMTNMDPVDTATYYCARHRPFYFDYWGQGTLVTVSS

SEQ ID NO: 105 - protein, 116 aa, Artificial Sequence
  Synthetic
QVTLKESGPVLVKPTETLTLTCTVSGFSLTSSGVSWVRQPPGKALEWIAAISSGGSTYYNSAFKSRLTISPDTSKSQVVLTMTNMDPVDTATYYCARHRPFYFDYWGQGTLVTVSS

SEQ ID NO: 106 - protein, 116 aa, Artificial Sequence
  Synthetic
QVTLKESGPVLVKPTETLTLTCTVSGFSLTSQGVSWVRQPPGKALEWIAAISSGGSTYYNSAFKSRLTISPDTSKSQVVLTMTNMDPVDTATYYCARHRPFYFDYWGQGTLVTVSS

SEQ ID NO: 107 - protein, 116 aa, Artificial Sequence
  Synthetic
QVTLKESGPVLVKPTETLTLTCTVSGFSLTSNAVSWVRQPPGKALEWIAAISSGGSTYYNSAFKSRLTISRDTSKSQVVLTMTNMDPVDTATYYCARHRPFYFDYWGQGTLVTVSS

SEQ ID NO: 108 - protein, 131 aa, Artificial Sequence
  Synthetic
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARIGESASDRYCSGGSCFGWFDPWGQGTLVTVSS

SEQ ID NO: 109 - protein, 106 aa, Artificial Sequence; identical to SEQ ID NO: 84
  Synthetic

SEQ ID NO: 110 - protein, 106 aa, Artificial Sequence
  Synthetic
DIQMTQSPSSLSASVGDRVTITCKGSQNIYKSLAWFQQKPGKVPKLLIYDANSLQTGIPSRFSGSGSGTDFTLTISSLQPEDVATYYCQQYYSGYTFGGGTKVEIK

SEQ ID NO: 111 - protein, 106 aa, Artificial Sequence
  Synthetic
DIQMTQSPSSLSASVGDRVTITCKGSQNIYKSLAWFQLKPGKVPKLLIYDANSLQTGIPSRFSGSGSGTDFTLTISSLQPEDVATYYCQQYYSGYTFGGGTKVEIK

SEQ ID NO: 112 - protein, 106 aa, Artificial Sequence
  Synthetic
DIQMTQSPSSLSASVGDRVTINCKGSQNIYKSLAWFQQKPGKVPKLLIYDANSLQTGIPSRFSGSGSGTDFTLTISSLQPEDVATYYCQQYYSGYTFGGGTKVEIK

SEQ ID NO: 113 - protein, 107 aa, Artificial Sequence
  Synthetic
DIQMTQSPSSLSASVGDRVTITCRASQGISNYLAWYQQKPGKVPKLLIYAASTLQSGVPSRFSGSGSGTDFTLTISSLQPEDVATYYCQKYNSAPPLFGGGTKVEIK

SEQ ID NO: 114 - protein, 118 aa, Artificial Sequence; identical to SEQ ID NO: 77
  Synthetic

SEQ ID NO: 115 - protein, 118 aa, Artificial Sequence
  Synthetic
QVTLKESGPVLVKPTETLTLTCTVSGFSLTSNGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 116 - protein, 118 aa, Artificial Sequence
  Synthetic
QVTLKESGPVLVKPTETLTLTCTVSGFSLTSNGVSWVRQPPGKALEWIAAISSGGTTYYNSAFKSRLSISRDTSKSQVVLTMTNMDPVDTATYYCARRYGYGWYFDFWGQGTLVTVSS

SEQ ID NO: 117 - protein, 118 aa, Artificial Sequence
  Synthetic
QVTLKESGPVLVKPTETLTLTCTVSGFSLTSSGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 118 - protein, 118 aa, Artificial Sequence
  Synthetic
QVTLKESGPVLVKPTETLTLTCTVSGFSLTSQGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 119 - protein, 118 aa, Artificial Sequence
  Synthetic
QVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 120 - protein, 106 aa, Artificial Sequence
  Synthetic
DIRMTQSPSLLSASVGDRVTLNCKASQNIYNSLAWYQQKLGEGPKVLIPNANSLQTGIPSRFSGSGSGTDFTLTISSLQPEDFATYFCQQFYSGYTFGAGTKLELK

SEQ ID NO: 121 - protein, 106 aa, Artificial Sequence
  Synthetic
DIQMTQSPSSLSASVGDRVTITCKASQNIYNSLAWYQQKPGKAPKVLIFNANSLQTGIPSRFSGSGSGTDFTLTISSLQPEDFATYYCQQFYSGYTFGQGTKLEIK

SEQ ID NO: 122 - protein, 106 aa, Artificial Sequence
  Synthetic
DIQMTQSPSSLSASVGDRVTITCKASQNIYNSLAWYQQKPGKAPKVLIFNANSLQTGVPSRFSGSGSGTDFTLTISSLQPEDFATYYCQQFYSGYTFGQGTKLEIK

SEQ ID NO: 123 - protein, 106 aa, Artificial Sequence
  Synthetic
DIQMTQSPSSLSASVGDRVTINCKASQNIYNSLAWYQQKPGKAPKVLIFNANSLQTGIPSRFSGSGSGTDFTLTISSLQPEDFATYYCQQFYSGYTFGQGTKLEIK

SEQ ID NO: 124 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRSFGQGTKLEIK

SEQ ID NO: 125 - protein, 14 aa, Artificial Sequence
  Synthetic
WVRQAPGKGLEWVA

SEQ ID NO: 126 - protein, 32 aa, Artificial Sequence
  Synthetic
RFTISRDNAKNSLYLQMNSLRAEDTAVYYCTR

SEQ ID NO: 127 - protein, 11 aa, Artificial Sequence
  Synthetic
WGQGTMVTVSS

SEQ ID NO: 128 - protein, 14 aa, Artificial Sequence
  Synthetic
WVRQAPGKRLEWVA

SEQ ID NO: 129 - protein, 15 aa, Artificial Sequence
  Synthetic
WYQQKPGQPPKLLIY

SEQ ID NO: 130 - protein, 32 aa, Artificial Sequence
  Synthetic
GVPDRFSGSGSGTDFTLTISSLQAEDVAVYYC

SEQ ID NO: 131 - protein, 10 aa, Artificial Sequence
  Synthetic
FGQGTKVEIK

SEQ ID NO: 132 - protein, 32 aa, Artificial Sequence
  Synthetic
GVPDRFTGSGSGTDFTLTISSLQAEDVAVYYC

SEQ ID NO: 133 - protein, 25 aa, Artificial Sequence
  Synthetic
QVTLKESGPVLVKPTETLTLTCTVS

SEQ ID NO: 134 - protein, 14 aa, Artificial Sequence
  Synthetic
WVRQPPGKALEWIA

SEQ ID NO: 135 - protein, 32 aa, Artificial Sequence
  Synthetic
RLTISRDTSKSQVVLTMTNMDPVDTATYYCAR

SEQ ID NO: 136 - protein, 11 aa, Artificial Sequence
  Synthetic
WGQGTLVTVSS

SEQ ID NO: 137 - protein, 32 aa, Artificial Sequence
  Synthetic
RLSISRDTSKSQVVLTMTNMDPVDTATYYCAR

SEQ ID NO: 138 - protein, 32 aa, Artificial Sequence
  Synthetic
RLTISRNTSKSQVVLTMTNMDPVDTATYYCAR

SEQ ID NO: 139 - protein, 10 aa, Artificial Sequence
  Synthetic
GFSLTSSGVS

SEQ ID NO: 140 - protein, 10 aa, Artificial Sequence
  Synthetic
GFSLTSQGVS

SEQ ID NO: 141 - protein, 10 aa, Artificial Sequence
  Synthetic
GFSLTSNAVS

SEQ ID NO: 142 - protein, 23 aa, Artificial Sequence
  Synthetic
DIQMTQSPSSLSASVGDRVTITC

SEQ ID NO: 143 - protein, 15 aa, Artificial Sequence
  Synthetic
WFQQKPGKVPKLLIY

SEQ ID NO: 144 - protein, 32 aa, Artificial Sequence
  Synthetic
GIPSRFSGSGSGTDFTLTISSLQPEDVATYYC

SEQ ID NO: 145 - protein, 10 aa, Artificial Sequence
  Synthetic
FGGGTKVEIK

SEQ ID NO: 146 - protein, 15 aa, Artificial Sequence
  Synthetic
WFQLKPGKVPKLLIY

SEQ ID NO: 147 - protein, 23 aa, Artificial Sequence
  Synthetic
DIQMTQSPSSLSASVGDRVTINC

SEQ ID NO: 148 - protein, 15 aa, Artificial Sequence
  Synthetic
WYQQKPGKAPKVLIF

SEQ ID NO: 149 - protein, 32 aa, Artificial Sequence
  Synthetic
GIPSRFSGSGSGTDFTLTISSLQPEDFATYYC

SEQ ID NO: 150 - protein, 10 aa, Artificial Sequence
  Synthetic
FGQGTKLEIK

SEQ ID NO: 151 - protein, 5 aa, Artificial Sequence
  Synthetic
SNGVS

SEQ ID NO: 152 - protein, 5 aa, Artificial Sequence
  Synthetic
SSGVS

SEQ ID NO: 153 - protein, 10 aa, Artificial Sequence
  Synthetic
RYGYGWYFDF

SEQ ID NO: 154 - protein, 32 aa, Artificial Sequence
  Synthetic
GVPSRFSGSGSGTDFTLTISSLQPEDFATYYC

SEQ ID NO: 155 - protein, 25 aa, Artificial Sequence [sequence omitted]

SEQ ID NO: 156 - protein, 118 aa, Artificial Sequence
  Synthesized
EVQLKESGPGLVQPSQTLSLTCTVSGFSLTSNGVSWVRQPPGKGLEWIAAISSGGTTYYNSAFKSRLSISRNTSKSQVLLKMNSLQTEDTAMYFCARRYGYGWYFDFWGPGTMVTVSS

SEQ ID NO: 157 - protein, 118 aa, Artificial Sequence
  Synthesized
EVTLKESGPVLVKPTETLTLTCTVSGFSLTSNGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 158 - protein, 118 aa, Artificial Sequence
  Synthesized
EVTLKESGPVLVKPTETLTLTCTVSGFSLTSNGVSWVRQPPGKALEWIAAISSGGTTYYNSAFKSRLSISRDTSKSQVVLTMTNMDPVDTATYYCARRYGYGWYFDFWGQGTLVTVSS

SEQ ID NO: 159 - protein, 118 aa, Artificial Sequence
  Synthesized
EVTLKESGPVLVKPTETLTLTCTVSGFSLTSSGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 160 - protein, 118 aa, Artificial Sequence
  Synthesized
EVTLKESGPVLVKPTETLTLTCTVSGFSLTSQGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 161 - protein, 118 aa, Artificial Sequence
  Synthesized
EVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 162 - DNA, 57 bp, Artificial Sequence
  Synthesized
atggagttcggcctgtcctggctgttcctggtggccatcctgaagggcgtgcagtgc

SEQ ID NO: 163 - protein, 19 aa, Artificial Sequence
  Synthesized
MEFGLSWLFLVAILKGVQC

SEQ ID NO: 164 - DNA, 57 bp, Artificial Sequence
  Synthesized
atggactggacctggagcatccttttcttggtggcagcagcaacaggtgcccactcc

SEQ ID NO: 165 - protein, 19 aa, Artificial Sequence
  Synthesized
MDWTWSILFLVAAATGAHS

SEQ ID NO: 166 - DNA, 66 bp, Artificial Sequence
  Synthesized
atggacatgcgcgtgcccgcccagctgctgggcctgctgatgctgtgggtgtccggctcctccggc

SEQ ID NO: 167 - protein, 22 aa, Artificial Sequence
  Synthesized
MDMRVPAQLLGLLMLWVSGSSG

SEQ ID NO: 168 - protein, 107 aa, Artificial Sequence [sequence omitted]

SEQ ID NO: 169 - protein, 106 aa, Artificial Sequence
  Synthesized
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 213 aa, Artificial Sequence
  Synthesized
DIQMTQSPSSLSASVGDRVTINCKASQNIYNSLAWYQQKPGKAPKVLIFNANSLQTGIPSRFSGSGSGTDFTLTISSLQPEDFATYYCQQFYSGYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 448 aa, Artificial Sequence
  Synthesized
QVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 448 aa, Artificial Sequence
  Synthesized
QVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 448 aa, Artificial Sequence
  Synthesized
EVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 448 aa, Artificial Sequence
  Synthesized
EVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 116 aa, Artificial Sequence
  Synthetic Peptide
QVTLKESGPVLVKPTETLTLTCTVSGFSLTSSGVSWVRQPPGKALEWIAAISSGGSTYYNSAFKSRLTISRDTSKSQVVLTMTNMDPVDTATYYCARHRPFYFDYWGQGTLVTVSS

SEQ ID NO: 179 - protein, 116 aa, Artificial Sequence
  Synthetic Peptide
QVTLKESGPVLVKPTETLTLTCTVSGFSLTSQGVSWVRQPPGKALEWIAAISSGGSTYYNSAFKSRLTISRDTSKSQVVLTMTNMDPVDTATYYCARHRPFYFDYWGQGTLVTVSS